(19) United States
(12) Patent Application Publication
(10) Pub. No.: US 2023/0364423 A1
(43) Pub. Date: Nov. 16, 2023

(54) Methods and Systems for Modifying Empathy by Modulating Type 2 Theta Oscillations

(71) Applicants: The Board of Trustees of Leland Stanford Junior University, Stanford, CA (US); Institute for Basic Science, Daejeon (KR)

(72) Inventors: Jin Hyung Lee, Palo Alto, CA (US); Hee-Sup Shin, Daejeon (KR)

(21) Appl. No.: 18/027,023

(22) PCT Filed: Sep. 30, 2021
(86) PCT No.: PCT/US2021/052997

§ 371 Date: Mar. 17, 2023

Related U.S. Application Data
(60) Provisional application No. 63/086,514, filed on Oct. 1, 2020.

(51) Int. Cl. A61N 1/36; A61N 1/05; A61N 5/06; A61K 31/46; A61K 31/407; A61P 25/00

(52) U.S. Cl. CPC A61N 1/36082; A61N 1/0534; A61N 5/0622; A61K 31/46; A61K 31/407; A61P 25/00

(57) ABSTRACT

The disclosure pertains to methods for modifying empathy in a subject, such as a human, by modulating type 2 theta oscillations in a brain region of the subject. The methods include increasing empathy in a subject, such as a human, by increasing type 2 theta oscillations in a brain region of the subject, thereby increasing empathy in the subject. The human subject may have a psychiatric or neurological condition that causes suboptimal empathy. Modulating type 2 theta oscillations in a brain region of a subject can be accomplished by optogenetic treatment, electric stimulation of a brain region, administration of a pharmaceutical drug, or a combination thereof. Also provided are systems for performing the methods disclosed herein.

### CROSS-REFERENCE TO RELATED APPLICATIONS

This application claims the benefit of U.S. Provisional Application No. 63/086,514, filed Oct. 1, 2020, the disclosure of which is incorporated herein by reference.

### INTRODUCTION

Empathy is an important function of human existence as social animals. Impairment of empathy is observed in numerous disorders, such as autism, depression, bipolar disorder, schizophrenia, anxiety disorder, attention-deficit/hyper-activity disorder, alexithymia, obsessive compulsive disorder, post-traumatic stress disorder, and psychopathy. Many neurological diseases including Alzheimer's disease and related dementia, Parkinson's disease, and epilepsy are also known to cause lack of empathy. One of the hallmark symptoms of addiction also includes lack of empathy. While empathy is a significant element of many of the psychiatric and neurological disorders, and addiction, little is known about why such common symptom of empathy impairment occurs. While each of the diseases show diverse profiles of symptoms, social deficits are important components that prevent the patients from carrying out normal life in a society. Even though the pathogenetic mechanisms for those diseases are thought to be diverse, the fact that many of those disease conditions induce social deficits suggest that some common neural mechanism might be shared among those neuro-psychiatric diseases. A common brain area or circuit critical for empathy may be affected in these disorders. Thus, many psychiatric or neurological conditions are associated with low levels of empathy even though the defining symptoms of such conditions are not directly related to empathy.

Humans and animals can acquire fear through observing conspecifics being subjected to aversive events, i.e. observational fear. Indeed, studies in primates and humans demonstrated that stronger vicarious fear response was positively associated with empathy. In observational fear learning in rodents, observing the demonstrator's distress responses can serve as a vicarious unconditional stimuli eliciting an association between the affective experience and the specific environmental context in the observer (context dependent conditioning). The cognitive process by which observing the demonstrator in distress induces a similar affective experience in the observer could be related to empathy. Using this rodent system many insights have recently been attained regarding the genes and circuits involved in affective empathy. For example, anterior cingulate cortex (ACC), basolateral amygdala (BLA), midline-thalamus, and insular regions that have been implicated in empathy in humans have also been confirmed to be involved in empathy in mice. These brain regions were also implicated in empathy in humans fMRI studies. Neurexin-3 molecules in somatostatin interneurons in ACC have been defined to gate the observational fear. The observational fear learning assay established in mice has made it possible to study the molecular, cellular, circuit mechanism underlying empathy.

Rodents display empathy-like behaviors such as observational fear learning, emotional contagion of pain, prosocial helping, or consolation of distressed others, suggesting that empathy is evolutionarily conserved in rodents as well as in humans. Particularly, observational fear learning has been utilized as a behavioral model for studying neurobiology of empathic fear in rodents. In this behavioral assay, a naïve animal is fear conditioned for the context of a chamber where it observed a conspecific animal receiving aversive treatments such as foot shocks in a neighboring chamber. Thus, in this conditioning, the unconditioned stimulus is a vicarious experience of the suffering of a demonstrator animal, unlike direct foot shocks used in the conventional fear conditioning. This social transfer of fear, an emotional contagion, to a naïve observer animal would require the ability of the observer to recognize the emotions of a demonstrator animal, suggesting that empathy is involved in this behavior as has been shown for observational fear in humans.

The capacity to share, appreciate, and respond to other's emotions has evolved over time, and ranges from primitive forms such as mimicry and emotional contagion to high-level forms such as perspective taking, sympathy, altruism, and targeted helping. Recent evidence shows that rodents possess a remarkable affective sensitivity to the emotional state of others and show diverse forms of empathy-like behaviors such as observational fear, emotional contagion of pain, consolation, and prosocial helping behaviors. Thus, rodent models are routinely used in the study of empathy in animals and humans alike. Also, these rodent models with appropriate assays can facilitate identification of underlying mechanisms for lack of sympathy and development of therapies to treat reduced or suboptimal empathy in humans.

### SUMMARY

The disclosure provides that synchronized type-2 theta oscillations in multi-regional neural circuits, for example, circuits involving medial septum, hippocampus, ACC, and/or BLA play a key role in cognitive process unique to vicarious, observational fear, and empathy. Accordingly, provided herein are methods for modulating empathy in a subject, such as a human subject. The methods comprise modulating type 2 theta oscillations in a brain region of the subject, thereby modulating empathy in the subject. In some cases, the methods comprise increasing type 2 theta oscillations in a brain region of the subject, thereby increasing empathy in the subject. The human subject may have a psychiatric or neurological condition that causes suboptimal empathy.

Modulating type 2 theta oscillations in a brain region of a subject can be accomplished by optogenetic treatment, electric stimulation of a brain region, administration of a pharmaceutical drug, or a combination thereof.

Also provided are systems for performing the methods disclosed herein.

### BRIEF DESCRIPTION OF THE DRAWINGS

FIGS. 1A-1S. A PLC-β1 deficiency in pyramidal neurons of the ACC impairs observational fear and excitability in layer 2/3 neurons. A: Diagram of the apparatus used for the observational fear task. B, C: Observational fear of PLC-β1^{-/-} (n=9) and wild-type (n=11) mice. PLC-β1 KO observers exhibited impaired observational fear conditioning (B) (F_{1, 18}=4.648, P<0.05, two-way repeated measures ANOVA followed by Student-Newman-Keuls post hoc test) and contextual memory 24 h after conditioning (C) (p<0.01, t(18)=3.213, t-7 test) compared with wild-type mice. D: Schematic of shRNA delivery into the bilateral ACC of a wild-type mouse brain. Bilateral injection of lentiviral vectors expressing shPLC-β1 or shSCR into the ACC of a mouse brain. E, F: shPLC-β1-injected observers (n=7) exhibited impaired observational fear conditioning (E) (F_{1,16}=4.916, P<0.05, two-way repeated measures ANOVA followed by Student-Newman-Keuls post hoc test) and contextual memory 24 h after conditioning (F) (p<0.01, t(16)=2.961, t-test) compared with shSCR-injected (n=11) mice. G: Schematic of bilateral injection of lentiviral vectors expressing shPLC-β1 or shSCR into the mPFC, including the PrL and IL, of a mouse brain. H, I: Similar freezing levels were seen during training (H) (F_{1, 12}=0.00406, P=0.950, two-way repeated measures ANOVA followed by Student-Newman-Keuls post hoc test) and in contextual memory 24 h after training (I) (p=0.824, t(12)=0.228, t-test) between shPLC-β1-injected (n=7) and shSCR-injected (n=7) mice (*p<0.05, **p<0.01, and ***p<0.001). All data are presented as means±SEM. J: Schematic of PLC-β1 deletion in excitatory neurons of the ACC. Unilateral injection of AAV5-CaMKIIα-GFP-Cre into the ACC of a PLC-β1loxP/loxP mouse. K, L: PLC-β1^{ACC/Cre} observers (n=9) exhibited impaired observational fear conditioning (K) (F_{1, 18}=10.981, p<0.01, two-way repeated measures ANOVA followed by Student-Newman-Keuls post hoc test) and contextual memory 24 h after conditioning (L) (p<0.05, t(18)=2.358, t-14 test) compared with PLC-β1ACC/GFP mice (n=11). M: Schematic of PLC-β1 knockdown in non-excitatory neurons in the ACC. Unilateral injection of AAV-loxP-U6-shPLC-β1-CMV-mCherry-loxP into the ACC of a CaMKIIα-Cre mouse. N, O: Similar freezing levels were found during observational fear conditioning (N) (F_{, 12}=0.162, P=0.695, two-way repeated measures ANOVA followed by Student-Newman-Keuls post hoc test) and in contextual memory 24 h after conditioning (O) (p=0.417, t(12)=−0.841, t-test) between ACC inhibitory neuron-specific shPLC-β1-injected (n=6) and shSCR-injected (n=8) observer mice. P: Representative traces of current-induced firings of layer 2/3 (upper panel) and 5/6 (lower panel) ACC neurons in PLC-β1^{+/+} (left panel) and PLC-β1^{-/-} (right panel) mice. Q: Average frequency firing ACC neurons from PLC-β1^{+/+} (n=52) and PLC-β1^{-/-} (n=48) mice with different current pulses. R: Average frequency of firings of layer 2/3 ACC neurons of PLC-β1^{+/+} (n=16) and PLC-β1^{-/-} (n=36) mice with different current pulses. S: Average frequency of firings of layer 5/6 ACC neurons of PLC-β1^{+/+} (n=20) and PLC-β1^{-/-} (n=28) mice with different current pulses. Data are presented as means±SEM (*p<0.05, ** p<0.01, and ***p<0.001).

FIGS. 2A-2O. Optogenetic inhibition of the reciprocal connections between the ACC and the BLA suppresses observational fear, not classical fear conditioning. A: Schematic diagram of NpHR3.0-mediated inhibition of ACC-BLA, BLA-ACC, and mPFC-BLA projections during the observational fear test. B: AAV5-CaMKIIα-NpHR3.0-eYFP (NpHR3.0-eYFP) was injected into the ACC of a wild-type mouse and the BLA was bilaterally illuminated with a yellow laser (left panel). Coronal section showing NpHR3.0-YFP-expressing excitatory neurons in the ACC and ACC fibers, imaged in the right or left BLA of an ACC-specific NpHR3.0-eYFP-injected mouse (right panel). C: Illumination of ACC inputs in the BLA during observational fear conditioning significantly reduced freezing level (F_{1, 19}=14.224, p=0.001, two-way repeated measures ANOVA followed by Student-Newman-Keuls post hoc test). D: NpHR3.0-eYFP-injected observers showed a decrease in 24-h contextual memory compared with AAV5-CaMKIIα-eYFP (eYFP)-injected mice (p<0.05, t(19)=−2.671, t-test). E: NpHR3.0-eYFP was injected into the BLA of a wild-type mouse, and the ACC was illuminated with a yellow laser left panel. Coronal section showing NpHR3.0-YFP-expressing excitatory neurons in the BLA and BLA fibers imaged in the ACC of a BLA-specific NpHR3.0-eYFP-injected mouse (right panel). F, G: Optogenetic inhibition of BLA inputs in the ACC of NpHR3.0-eYFP-injected observers during observational fear conditioning significantly reduced observational fear conditioning (F) (F1, 21=12.858, P<0.01, two-way repeated measures ANOVA followed by Student-Newman-Keuls post hoc test) and 24-h contextual memory (G) (p<0.05, t(21)=−2.251, t-test) compared with eYFP-injected mice. H: NpHR3.0-eYFP was injected into the mPFC, including the PrL and IL, of a wild-type mouse and the BLA was bilaterally illuminated with a yellow laser (left panel). Coronal section showing NpHR3.0-YFP-expressing excitatory neurons in the mPFC and mPFC fibers imaged in the right or left BLA of an ACC-specific NpHR3.0-eYFP-injected mouse (right panel). I: Illumination of mPFC inputs to the BLA during observational fear conditioning had no effect on observational fear conditioning (F_{1, 13}=0.00232, P=0.962, two-way repeated measures ANOVA followed by Student-Newman-Keuls post hoc test). J: There was no significant difference in 24-h contextual memory between NpHR3.0-eYFP-injected observers and eYFP-injected mice (p=0.267, t(13)=1.160, t-test). K: AAV5-CaMKIIα-NpHR3.0-eYFP (NpHR3.0) was injected into the ACC of a wild-type mouse and the BLA was illuminated with a yellow laser. L: During contextual fear conditioning, illumination of ACC inputs to the BLA had no significant effect on freezing behavior during the training period (F_{1, 12}=0.0587, P=0.001, two-way repeated measures ANOVA followed by Student-Newman-Keuls post hoc 1 test). M: There was no significant difference in 24-h contextual memory between NpHR3.0-eYFP-injected observer mice and AAV5-CaMKIIα-eYFP (eYFP)-injected mice (p=0.787, t(12)=0.276, t-test). N, O: During contextual fear conditioning, optogenetic inhibition of BLA inputs in the ACC of NpHR3.0-eYFP-injected observers had no effect on freezing behavior during the training period (N) (F_{1, 12}=1.398, p=0.260, two-way repeated measures ANOVA) or 24-h contextual memory (O) (p=0.698, t(12)=0.398, t-test) compared with eYFP-injected mice.

FIG. 3A-3L. The 4-8 Hz oscillation in the ACC-BLA circuit is absent in the PLC-β1 knock-down mice during observational fear. A: Schematic diagram showing simultaneous recording of LFPs in the ACC and BLA of freely behaving ACC-specific PLC-β1-knockdown observers during observational fear. B: Schematic diagram showing LFP recordings during observational fear; recordings from habituation and inter-shock periods of the observational fear conditioning protocol were used for each observer mouse. C: Representative original traces of LFP recordings in the ACC and BLA of shSCR-injected (upper) and ACC-specific PLC-β1-knockdown (bottom) observers during habituation. D: Same as C during conditioning. E, F: Colorized power spectra of ACC-specific shSCR traces (E) and ACC-specific shPLC-β1 traces (F) shown in c (habituation) and d (conditioning). Note the substantially increased power of theta rhythms in the ACC and BLA of ACC-specific shSCR-injected mice versus the decreased power of these rhythms in ACC-specific PLC-β1 knockdown observers during the conditioning period compared with the habituation period. G, H: Averaged power spectrum density of neuronal activities in the ACC (G) and the BLA (H) of shSCR-injected mice (n=7) during habituation and conditioning periods. I, J: Averaged power spectrum density of neuronal activities in the ACC (I) and the BLA (J) of ACC-specific PLC-β1 knockdown observers (n=10). K: Averaged cross-correlograms between the ACC and the BLA for ACC-specific shSCR-injected observers during habituation and conditioning periods. L: Same as K for ACC-specific PLC-β1 knockdown observers. Significant p-values for rank-sum tests on lags between habituation and conditioning correlations for each experimental group are shown under the traces.

FIGS. 4A-4T. Attenuation of type-2 hippocampal theta rhythms by optogenetic inhibition of septo-hippocampal GABAergic projections decreases observational fear and 4-8 Hz theta 2 rhythms in the ACC and the BLA. A: AAV5-EF1α-DIO-NpHR3.0-eYFP (DIO-NpHR) was injected into the MS of PV-Cre transgenic mice, and the dorsal fornix was illuminated with a yellow laser (left panel). eYFP expression in the MS and eYFP-expressing fornix fibers imaged in the dorsal fornix of MS-specific DIO-NpHR-injected PV-Cre transgenic mice (right panel). B: EEG electrodes were implanted into the ACC, BLA, and hippocampus of PV-Cre transgenic mice. C: Schematic diagram of NpHR3.0-mediated inhibition of MS GABAergic projections to the hippocampus (MSGABA-Hipp) in observational fear tests. D, E: Optogenetic inhibition of GABAergic MS inputs to the hippocampus during observational fear conditioning significantly reduced freezing level (D) (F_{1, 22}=17.277, p<0.001, two-way repeated measures ANOVA followed by Student-Newman-Keuls post hoc test). DIO-NpHR-injected observers showed a decrease in 24-h contextual memory compared with AAV5-EF1α-DIO-eYFP (DIO-eYFP)-injected mice (E) (p<0.05, t(21)=2.480, t-test). F: Schematic diagram of NpHR3.0-mediated inhibition of MSGABA-Hipp projections in classical contextual fear conditioning tests. G, H: Optogenetic inhibition of GABAergic MS inputs to the hippocampus of DIO-NpHR-injected observers during contextual fear conditioning had no effect on freezing level during the training period (G) (F_{1,11}=0.00289, p=0.958) and on 24-h contextual memory (H) (p=0.817, t(11)=0.238, t-test) compared with DIO-eYFP-injected mice. I-K: Averaged power spectrum density of neuronal activities in the ACC (I), BLA (J), and hippocampus (K) of DIO-eYFP-injected observers (n=7). L-N: Averaged power spectrum density of neuronal activities in the ACC (L), BLA (M), and hippocampus (N) of DIO-NpHR-injected observers (n=6). O-Q: Changes in LFP power from habituation to conditioning at frequency ranges of 4-8 Hz in the ACC (O; p<0.05, t(11)=−29678, t-test), the BLA (P; p<0.01, t(11)=−3.1829, t-test) and the hippocampus (Q; p<0.05, t(11)=−2.4869, t-test). R-T: Changes in LFP power from habituation to conditioning at frequency ranges of 8-12 Hz in the ACC (R; p=0.3189, t(11)=1.0439, t-test), BLA (S; p=0.3061, t(11)=1.0734, t-test), and hippocampus (T; p=0.1444, t(11)=1.5716, t-test).

FIGS. 5A-5J. The power modulation in type-2 theta in the hippocampus-cingulate-amygdala circuit is temporally coupled with freezing bouts, and the degree of this power modulation predicts the magnitude of observational fear. A, C: Averaged spectrograms of the ACC, BLA and Hipp, centered on the onset (A) and offset (C) of freezing behavior. B, D: Averaged z-scores of LFP power in the ACC, BLA, and Hipp around freezing onset (B) and offset (D). Total 42 (A and B) and 41 epochs from 7 mice (C and D) were used, respectively. Black lines on spectrograms indicate changes in the motion index. E-G: Pearson's correlation analysis of freezing behavior during observational fear versus averaged changes in the power of the type-2 theta rhythm in the ACC (E), BLA (F), and hippocampus (Hipp, G). H-J: Pearson's correlation analysis of freezing behavior during observational fear versus averaged changes in the power of type-1 theta rhythm in the ACC (H), BLA (I), and hippocampus (J).

FIGS. 6A-6W. Optogenetic enhancement of hippocampal type-2 theta modulations observational fear. A: AAV5-EF1α-DIO-ChR2-eYFP (DIO-ChR2) was injected into the MS of PV-Cre transgenic mice and the dorsal fornix was illuminated with a blue laser (left panel). eYFP expression in the MS and eYFP-expressing fornix fibers, imaged in the dorsal fornix of MS-specific DIO-ChR2-injected PV-Cre transgenic mice (right panel). B: Three EEG electrodes were implanted each into the ACC, BLA, and hippocampus of PV-Cre transgenic mice. C: Schematic diagram of ChR2-mediated activation of septo-hippocampal GABAergic projection in the observational fear test. D, E: Optogenetic activation of GABAergic MS inputs to the hippocampus during observational fear conditioning increased freezing level (D) (F_{1,17}=9.155, p=0.008, two-way repeated measures ANOVA followed by Student-Newman-Keuls post hoc test). No difference in the 24-h contextual memory, however, was observed between the test and the control group (E) (p=0.587, t(17)=−0.554). F-H: Averaged power spectrum density of LFP activities in the ACC (F), BLA (G), and hippocampus (H) of DIO-eYFP-injected, control, observers (n=8). I-K: Averaged power spectrum density of LFP activities in the ACC (I), the BLA (J), and the hippocampus (K) of DIO-ChR2-injected, test, observers (n=11). L-N: Changes in the LFP power from habituation to conditioning at the frequency range of 4-8 8 Hz in the control (blue bar) and the test group (red bar) in the ACC (L; p<0.05, t(17)=2.4211, t-test), BLA (M; p=0.1976, t(17)=1.341, t-test) and hippocampus (N; p<0.05, t(17)=2.3425, t-test). O-Q: Pearson's correlation analysis of freezing behavior of a mouse during observational fear versus averaged power change in the type-2 theta rhythm in the ACC(O), BLA (P), and hippocampus (Hipp, Q). R-W: Altered type-1 theta rhythm by optogenetic activation of septo-hippocampal GABAergic projection was not related to observational fear. Changes in LFP power from habituation to conditioning at frequency ranges of 8-12 Hz in the ACC (R; p=0.3162, t(17)=−1.0327, t-test), BLA (S; p<0.01, t(17)=−3.3174, t-test), and hippocampus (Hipp, T; p<0.05, t(17)=−2.2106, t-test). Pearson's correlation analysis of freezing behavior during observational fear versus averaged changes in the power of type-1 theta rhythm in the ACC (U), BLA (V), and hippocampus (W).

### DETAILED DESCRIPTION

Neurons in the central nervous system can activate in oscillatory patterns. These oscillations can be categorized by the frequency of oscillation. For example, theta oscillations occur in the 4 Hz to 8 Hz range whereas alpha oscillations occur in the 8 Hz to 15 Hz range. Theta oscillations include type 1 theta oscillations and type 2 theta oscillations. In some cases, type 1 oscillations occur between 7 Hz and 8 Hz. Type 1 oscillations sometimes occur during voluntary motion and REM sleep. Type 1 oscillations will usually not be affected by administration of the pharmaceutical drug atropine. Type 2 oscillations sometimes occur between 4 Hz and 7 Hz. Type 2 oscillations sometimes occur while the subject is anesthetized, such as by administration of urethane (i.e. ethyl carbamate). Type 2 oscillations sometimes also occur when the subject is immobilized due to fear, e.g. when a laboratory rat is fearful of a nearby cat or ferret. Type 2 oscillations sometimes occur briefly in the moments while an animal is preparing to move, but has not yet moved. The type 1 and type 2 notation is sometimes applied to oscillations in the hippocampus.

The disclosure provides that synchronized type-2 theta oscillations in multi-regional neural circuits, for example, circuits involving medial septum, hippocampus, ACC, and/or BLA play a key role in cognitive process unique to vicarious, observational fear, and empathy. Accordingly, provided herein are methods for modifying, particularly, increasing empathy in a subject, such as a human subject. The methods comprise modulating type 2 theta oscillations in a brain region of the subject, thereby modifying empathy in the subject.

In some cases, the methods comprise increasing type 2 theta oscillations in a brain region of the subject, thereby increasing empathy in the subject. The human subject may have a psychiatric or neurological condition that causes suboptimal empathy.

Modulating type 2 theta oscillations in a brain region of a subject can be accomplished by optogenetic treatment, genetic modification, electric stimulation of a brain region, administration of a pharmaceutical drug, or a combination thereof.

Also provided is a system for performing the methods disclosed herein.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a,” “an,” and “the” include plural referents unless the context clearly dictates otherwise. Thus, for example, reference to “a discrete entity” includes reference to one or more discrete entities. It is further noted that the claims may be drafted to exclude any element, e.g., any optional element. As such, this statement is intended to serve as antecedent basis for use of such exclusive terminology as “solely,” “only,” and the like in connection with the recitation of claim elements, or the use of a “negative limitation.”

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

“Suboptimal empathy” as used herein refers to an emotional trait in a subject characterized by the level of empathy that is lower than empathy observed in an average subject of that species. Suboptimal empathy refers to the inability to recognize or identify with the feelings and needs of others and can be exhibited by, for example, callous and unemotional attitude, inability to recognize distress in other humans or animals, and inability to infer emotions of other humans or animals.

Empathy can be assessed in various manners. Exemplary measures or types of empathy include observational fear learning, emotional contagion of pain, prosocial helping, consolation of distressed others, perspective taking, sympathy, altruism, and targeted helping. The term “suboptimal empathy” includes, for example, when a medical or psychiatric professional determines that the subject has a level of empathy that is below the average level of empathy for an individual in society. “Suboptimal empathy” also includes situations wherein a medical or psychiatric professional determines that the subject has a deficit in empathy that causes interpersonal problems.

Certain tests are used in psychology to assess empathy in a human. For example, an article by Carré et al. (2013), Psychol Assess; 25(3):679-91, describes certain empathy scales used to assess empathy in humans. The Carré et al. article is incorporated herein in its entirety. Another such method is described by Jolliffe et al. in the article “Development and validation of the Basic Empathy Scale,” Journal of Adolescence, Volume 29, Issue 4, August 2006, Pages 589-611, which is herein incorporated by reference in its entirety. Thus, a person of ordinary skill in the art can determined whether a subject, particularly, a human, has suboptimal empathy, i.e., the level of empathy that is lower than empathy observed in an average human.

The term “Theta oscillations” is used interchangeably with “theta waves” or “theta rhythms” to refer to neural oscillations with a frequency ranging from 4 Hz to 8 Hz. The term “neural oscillations” is used interchangeably herein with the terms “brainwave” and “brain wave.” Type 2 theta oscillations is used interchangeably with type-2 theta oscillations. Theta oscillations is used interchangeably with theta rhythms. The terms subject and patient are used interchangeably herein to refer to an animal, e g a human.

A “therapeutically effective amount,” a “therapeutically effective dose,” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of a compositions is an amount that is sufficient, when administered to the individual, to palliate, ameliorate, stabilize, reverse, prevent, slow or delay the progression of a disease state or condition present in the subject.

As used herein, the terms “determining,” “measuring,” “assessing,” and “assaying” are used interchangeably and include both quantitative and qualitative determinations.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal, intramuscular, subcutaneous, and the like.

### Methods

The disclosure suggest that the type-2 hippocampal theta oscillations induce a long-range neuronal network coupling between the hippocampus, ACC, and BLA, and then drives, in mice, affective empathy underlying observational fear without affecting classical fear conditioning. This disclosure highlights the translational potential of the type-2 hippocampal theta oscillations as a treatment for suboptimal empathy, for example, suboptimal empathy caused by diverse neuropsychiatric or neurological conditions.

Accordingly, in some cases, provided are methods of modifying empathy in a human subject by modulating type 2 theta oscillations in a brain region of the subject, thereby modifying empathy in the subject. In some cases, the methods comprise increasing empathy in a human subject having by increasing type 2 theta oscillations in a brain region of the subject, thereby increasing empathy in the subject. In some cases, the subject has suboptimal empathy, which may be caused by a psychiatric or neurological condition.

Modulating type 2 theta oscillations can be accomplished by, for example, optogenetic treatment, electrical stimulation of a brain region using one or more electrodes, administering a pharmaceutical drug, genetically modifying neurons in the brain region to directly modulation the oscillations, or a combination thereof. In some cases, the modulation involves increasing the type 2 theta oscillations. In other cases, the modulation involves decreasing type 2 theta oscillations.

Modulating type 2 theta oscillations can also be accomplished by sensory stimulation, electrical stimulation, mechanical stimulation, or mechanically activated genetically targeted stimulation, all with or without entrainment, gene therapy, cell therapy, and pharmaceutical drugs.

### Optogenetic Treatment

Optogenetic treatment includes genetic modification of cells in certain brain regions and/or cells of certain types followed by contacting the genetically modified brain region with light. The genetic modification causes the modified cells to express light-sensitive ion channels. Contacting the cells, such as neurons, with light activates these channels, influencing the activation of the cells, such as neurons.

In some cases, optogenetic treatment is administered with entrainment. In some cases, optogenetic treatment is administered without entrainment.

Certain details of genetically modifying cells in the brain, such as neurons in specific brain area or other non-neuronal cell types are described in the article by Haery et al. (2019), Frontiers in Neuroanatomy, Vol. 13, Article 93, which is herein incorporated by reference in its entirety.

In some cases, neurons or other brain cells are genetically modified by delivering a gene via a viral vector, such as adenoviral vector, adeno-associated viral vector, or a lentiviral vector. The viral vector comprises a gene that encodes for a light-sensitive ion channel. Non-limiting examples of such light-sensitive ion channels include opsins, for example, channelrhodopsin, such as channelrhodopsin2, algal channelrhodopsin, or microbial rhodopsin. Additional examples of light-sensitive ion channels are described in U.S. Patent Application Publication numbers 2020/0121746 and 2020/0191776, which are incorporated by reference herein in their entirety.

In some cases, the vector, such as a viral vector, containing the gene encoding for the light-sensitive ion channel is injected specifically into the region of interest thereby limiting the expression of the light-sensitive ion channel in the targeted area within the brain. In some cases, the viral vector is injected into one or more of: hippocampus, septo-hippocampus, ACC, BLA, midline thalamus, insulate regions, MS, fimbria fornix. In one embodiment, the viral vector is injected only into the thalamus and the virus transfects cells in a larger area than just the thalamus, including, for example, ACC.

Alternatively, the viral vector can contain the gene encoding the light-sensitive ion channel under the control of a specific regulatory element, such as a promoter or enhancer, which induces the expression of the light-sensitive ion channel in specific and desired area or cell types. Certain such promoters or enhancers are described by Haery et al., particularly, in Table 4. Additional such promoters or enhancers are described by an article by Mich et al. (2021), Cell Rep.; 34(13): 108754, which is herein incorporated by reference in its entirety.

Certain such promoters or enhancers include promoters for: human synapsin1, MeCP2, neuron-specific enolase, BM88, mDLX, dopamine beta-hydroxylase, PRSx8 (modified dopamine beta-hydroxylase), PCP2, FEV, melanin-concentrating hormone, SLC6A4, NR2E1, GfABC1D, Aldh1/1, myelin-associated glycoprotein, ICAM-2, CLDN5, Tie-2, and FLT1.

The brain region that is specifically modified to express light-sensitive ion channel can be one or more of: hippocampus, septo-hippocampus, ACC, BLA, midline thalamus, insulate regions, medial septum (MS), fimbria fornix. In some cases, the brain region is the hippocampus. In some cases, the brain region is the septo-hippocampus. In some cases the brain region is the ACC. In some case, the brain region is the BLA. In some cases, the brain region is the medial septum. In some cases the brain region is the fimbria fornix.

In some cases of the optogenetic treatment, the method involves GABAergic neurons. These neurons are influenced by the neurotransmitter gamma-aminobutyric acid (GABA). Thus, in some cases the method involves neurons that include a synapse that is responsive to GABA. In some cases, the GABAergic neurons are located in the fimbria fornix. In some cases, the GABAergic neurons are located in the septo-hippocampus.

After the brain region specific or brain cell specific expression of the light-sensitive ion channels is established, the specific area can be stimulated to generate type-2 theta oscillations. This can be achieved by illuminating the targeted brain area using a laser. Such laser can be surgically placed inside the brain of a subject and activated via a wired or wireless communication to illuminate the target area. Such illumination of the target area activates the light-sensitive ion channels in the target area thereby inducing type-2 theta oscillations.

Wireless and even battery-free devices for activating lasers installed within the brain of a subject are known in the art. Certain such devices are described by an article by Won et al. (2021), Wireless and battery-free technologies for neuroengineering, Nat Biomed Eng, which is herein incorporated by reference in its entirety.

A power source and a controlling device can be connected to the installed lasers. Such connections can be wired or wireless. The controlling device can be configured to power the lasers installed in the brain of a subject to induce type-2 theta oscillations.

Additional details of the optogenetic stimulation of the target area within a brain are known to a person of ordinary skill in the art and such embodiments are within the purview of the invention.

### Electrical Stimulation Treatment

In some cases, the method comprises electrical stimulation of a brain region using one or more electrodes to induce type-2 theta oscillations. In some cases, the electrodes can be positioned, either temporarily or permanently, at the brain region of interest. Typically, such techniques are called as “deep brain stimulation.”

Wireless and even battery-free devices for activating electrodes installed within the brain of a subject are known in the art. Certain such devices are described by an article by Won et al. mentioned above, which is herein incorporated by reference in its entirety.

The brain regions of interest can be as described above and include one or more of: hippocampus, septo-hippocampus, ACC, BLA, midline thalamus, insulate regions, MS, fimbria fornix. In some cases, the brain region is the hippocampus. In some cases, the brain region is the septo-hippocampus. In some cases the brain region is the ACC. In some case, the brain region is the BLA. In some cases, the brain region is the medial septum. In some cases the brain region is the fimbria fornix.

Such electrodes can be surgically placed inside the brain of a subject and it can be activated via a wired or wireless communication to electrically stimulate the target area. Such electrical stimulation of the target area induces type-2 theta oscillations in the area of interest. Won et al. identified above provides information relevant to some of these embodiments and such embodiments are within the purview of the disclosure.

### Pharmaceutical Drugs

In some cases, the method involves administration of a pharmaceutical drug. In some cases, the pharmaceutical drug is an anticholinesterase. Anticholinesterases are drugs increase acetylcholine existence after release from cholinergic nerve endings. Anticholinesterases may inhibiting both acetylcholinesterase and butyrylcholinesterase. The prosthetic type of anticholinesterases inhibit the anionic site of acetylcholinesterase. Acid-transferring anticholinesterases react with the enzyme and form an intermediate compound that cannot be hydrolyzed as rapidly as the acetylated enzyme formed from acetylcholine.

In some cases, the drug is an anticholinesterase agent is atropine. In some cases, the drug is physostigmine, which is also known as eserine.

In some cases the drug is an activator of PLC, particularly, an activator of PLC-β1 and/or PLC-β4. In some cases, a PLC activator is m3M3FBS (2,4,6-trimethyl-N-(meta-3-trifluoromethyl-phenyl)-benzenesulfonamide), or thapsigargin. Additional examples of activators of PLC are described in U.S. Patent Application Publication No. 20200306213 and 20110123994, which are incorporated herein in their entireties.

In some cases, the pharmaceutical drug is modified and the target brain regions are modified in such a manner that the pharmaceutical drug only acts on the target brain regions. Certain such methods are disclosed in the article Shields et al. (2017), Science, 356(6333), which is incorporated by reference in its entirety.

As described in Shields et al., in some cases, the drug activity is restricted by tethering in a molecule called Drug Acutely Restricted by Tethering (DART). DART is targeted to a type of cell that expresses an enzyme that converts DART to active drug. Specific cells within the brain of a subject can be modified to express the enzyme that acts on DART. DART can be delivered to the cells expressing the enzyme, which in turn delivers the drug to the target cells.

### Genetic Modifications of the Target Neurons

In some cases, the methods comprise genetically modifying neurons in the brain region to directly modulate the oscillations. Thus, these embodiments do not require contacting the neurons with light, but instead involve modifying type 2 theta oscillations through other biochemical routes. For example, such modulating involves genetically modifying cells, such as neurons in target brain region to directly modulate type-2 theta oscillations. Unlike optogenetic treatment, this option modifies type 2 theta oscillations through other biochemical routes.

For example, Arshaad et al. (2021), Scientific Reports, volume 11, Article number: 1099 show that inhibition of T-type Ca²⁺ channels Caᵥ3.2 increase type-2 theta oscillations in hippocampal region. Thus, in one embodiment, modulating type 2 theta oscillations comprise modulating Caᵥ3.2 expression in certain brain regions, particularly, ACC, BLA, medial septum, and/or hippocampus. In some cases, type 2 theta oscillations are increased by decreasing Caᵥ3.2 expression in certain brain regions, particularly, ACC, BLA, medial septum, and/or hippocampus. Such decrease can be achieved by genetically modifying the cells in one or more of these regions to express an inhibitory RNA that inhibits the expression of the gene encoding Caᵥ3.2.

In some cases, modulating type 2 theta oscillations comprise modulating Caᵥ3.1 expression in certain brain regions, particularly, ACC, BLA, medial septum, and/or hippocampus. In some cases, type 2 theta oscillations are increased by decreasing Caᵥ3.1 expression in certain brain regions, particularly, ACC, BLA, medial septum, and/or hippocampus. Such decrease can be achieved by genetically modifying the cells in one or more of these regions to express an inhibitory RNA that inhibits the expression of the gene encoding Caᵥ3.1.

In some cases, modulating type 2 theta oscillations comprise modulating CAV3 gene expression in certain brain regions, particularly, ACC, BLA, medial septum, and/or hippocampus. CAV3 gene encodes for the protein Caveolin-3. In some cases, type 2 theta oscillations are increased by decreasing CAV3 expression in certain brain regions, particularly, ACC, BLA, medial septum, and/or hippocampus. Such decrease can be achieved by genetically modifying the cells in one or more of these regions to express an inhibitory RNA that inhibits the CAV3 gene.

In some cases, modulating type 2 theta oscillations comprise modulating PCF-β1 expression in certain brain regions, particularly, ACC, BLA, medial septum, and/or hippocampus. In some cases, type 2 theta oscillations are increased by increasing PCF-β1 expression in certain brain regions, particularly, ACC, BLA, medial septum, and/or hippocampus. Such decrease can be achieved by genetically modifying the cells in one or more of these regions to express a gene encoding PCF-β1.

In some cases, modulating type 2 theta oscillations comprise modulating PCF-β4 expression in certain brain regions, particularly, medial septum. In some cases, type 2 theta oscillations are increased by increasing PCF-β4 expression in certain brain regions, particularly, ACC, BLA, medial septum, and/or hippocampus. Such decrease can be achieved by genetically modifying the cells in one or more of these regions to express the gene encoding PCF-β4.

### Subjects and Brain Regions Specification Activation of Type-2 Theta Oscillations Subjects

The method can be used with any subject determined to have suboptimal empathy. In some cases, the subject has a psychiatric or neurological condition is selected from the group consisting of: autism spectrum disorder, dementia, addiction, depression, anxiety, bipolar disorder, schizophrenia, attention-deficit hyperactivity disorder (ADHD), alexithymia, obsessive-compulsive disorder (OCD), post-traumatic stress disorder (PTSD), psychopathy, Parkinson's disease, and epilepsy. In some cases, the psychiatric or neurological condition is selected from the group consisting of autism spectrum disorder, dementia, and addiction. Autisms spectrum disorders include autism and Aspberger syndrome. Exemplary types of dementia include Alzheimer's disease, vascular dementia, dementia with Lewy bodies, and frontotemporal dementia. Attention-deficit hyperactivity disorder (ADHD) is used interchangeably herein with attention-deficit disorder (ADD). Exemplary addictions include alcohol, cannabis, nicotine, opioids, hallucinogens, and stimulants.

The particular brain region which is modulated can influence the efficacy of the treatment. In some cases, the brain region can be: hippocampus, septo-hippocampus, ACC, BLA, midline thalamus, insulate regions, medial septum, fimbria fornix, or a combination of any of one or more of any of these regions. In some cases, the brain region is the hippocampus. In some cases, the brain region is the septo-hippocampus. In some cases the brain region is the ACC. In some case, the brain region is the BLA. In some cases, the brain region is the medial septum. In some cases the brain region is the fimbria fornix. In some cases, two or more of the listed brain regions are modulated.

In some cases, the brain region comprises pyramidal neurons of the ACC, medial prefrontal cortex, and/or septo-hippocampal GABAergic projections.

### Systems

Provided are systems for modulating empathy in a subject, such as humans by modulating type 2 theta oscillations in certain brain regions of a subject. In some cases, the systems increase empathy in a subject, such as humans having suboptimal empathy, by increasing type 2 theta oscillations in certain brain regions of the subject. The suboptimal empathy can be caused by a psychiatric or neurological condition involving suboptimal empathy. In some cases, the system includes a device configured to modulation type 2 theta oscillations in a brain region of the subject.

In some cases, the device is an optogenetic device. The optogenetic device includes a light source operationally coupled to a light transmitter configured and positioned to deliver light to the brain region. For example, for optogenetic treatment the subject can have neurons in their brain genetically altered to express a light sensitive ion channel, thereby allowing for contacting of light onto the cells with those channels. Certain details of genetically modifying cells in the brain to express a light-sensitive ion channels are described above and such details are also relevant to the systems disclosed herein.

If the cells are neurons, then exposing cells expressing light-sensitive ion channels to light affects the firing of the neurons. In some cases, the light transmitter includes a fiber optic cable. In some cases, the end of the fiber optic cable is positioned in the brain region so that light is directed towards the optogenetically modified neurons in that brain region.

In some cases, the device is an electrical stimulation device including an electrical current generator operationally coupled to at least one electrode configured and positioned to deliver electrical stimulation to the brain region. In some cases, the system comprises two electrodes. In some cases, the system comprises more than two electrodes.

In some cases, the brain region is selected from the group consisting of: hippocampus, septo-hippocampus, ACC, BLA, midline thalamus, insulate regions, MS, fimbria fornix, and amygdala.

Notwithstanding the appended claims, the disclosure is also defined by the following clauses:

Clause 1. A method of modulating empathy in a human, comprising: modulating type 2 theta oscillations in a brain region of the human.

Clause 2. The method of clause 1, wherein modulating empathy comprises increasing empathy in the human, wherein the human has suboptimal empathy.

Clause 3. The method of clause 2, wherein the suboptimal empathy in the human is caused by a psychiatric or neurological condition.

Clause 4. The method of any of preceding clauses, wherein modulating type 2 theta oscillations comprises optogenetic treatment with or without entrainment.

Clause 5. The method of any of preceding clauses, wherein the optogenetic treatment comprises stimulating GABAergic neurons.

Clause 6. The method of clause 5, wherein the GABAergic neurons are located at the fimbria fornix.

Clause 7. The method of clause 5 or 6, wherein the GABAergic neurons are located at the septo-hippocampus.

Clause 8. The method of any of clauses 1 to 3, wherein modulating type 2 theta oscillations comprises genetically modifying neurons in the brain region to directly modulate type-2 theta oscillations.

Clause 9. The method clause 8, wherein genetically modifying neurons in the brain region comprises modifying the expression of one or more genes encoding Caᵥ3.2, Caᵥ3.1, CAV3, PLC-β1, and PLC-β4.

Clause 10. The method of any of clauses 1 to 3, wherein modulating type 2 theta oscillations comprises electrical stimulation of the brain region using one or more electrodes.

Clause 11. The method of any of clauses 1 to 3, wherein the modulating comprises administering to the human a pharmaceutical drug.

Clause 12. The method of clause 11, wherein the pharmaceutical drug is an anticholinesterase agent.

Clause 13. The method of clause 12, wherein the anticholinesterase agent is atropine or physostigmine.

Clause 14. The method of clause 11, wherein the pharmaceutical drug is an activator of PLC.

Clause 15. The method of clause 14, wherein the activator of PLC is an activator of PLC-β1 and/or PLC-β4.

Clause 16. The method of clause 14 or 15, wherein the activator of PLC is m3M3FBS (2,4,6-trimethyl-N-(meta-3-trifluoromethyl-phenyl)-benzenesulfonamide) or thapsigargin.

Clause 17. The method of any of clauses 1 to 16, wherein the brain region is: hippocampus, septo-hippocampus, anterior cingulate cortex (ACC), basolateral amygdala (BLA), midline thalamus, insulate regions, medial septum (MS), or fimbria fornix.

Clause 18. The method of any of clauses 1 to 17, wherein the type 2 theta oscillations are synchronized in the hippocampus, ACC, or BLA.

Clause 19. The method of clause 3, wherein the psychiatric or neurological condition is Alzheimer's disease, autism spectrum disorder, dementia, addiction, depression, anxiety, bipolar disorder, schizophrenia, attention-deficit hyperactivity disorder (ADHD), alexithymia, obsessive-compulsive disorder (OCD), post-traumatic stress disorder (PTSD), psychopathy, Parkinson's disease, or epilepsy.

Clause 20. The method of clause 19, wherein the psychiatric or neurological condition is selected from the group consisting of: autism spectrum disorder, dementia, and addiction.

Clause 21. The method of any of clauses 1 to 20, wherein the modulating is increasing type 2 theta oscillations.

Clause 22. The method of clause 1, wherein the modulating is decreasing type 2 theta oscillations.

Clause 23. The method of any of clauses 1 to 3, wherein the modulating of type 2 theta oscillations does not induce modulation of type 1 theta oscillations.

Clause 24. A system for modulating empathy in a human, comprising: a device configured to modulate type 2 theta oscillations in a brain region of the human.

Clause 25. The system of clause 24, wherein the device is configured to increase empathy in a human having suboptimal empathy.

Clause 26. The system of clause 24 or 25, wherein the device is an optogenetic device comprising a light source operationally coupled to a light transmitter configured and positioned to deliver light to the brain region.

Clause 27. The system of clause 24 or 25, wherein the device is an electrical stimulation device comprising an electrical current generator operationally coupled to at least one electrode configured and positioned to deliver electrical stimulation to the brain region.

Clause 28. The system of clause 27, wherein the system comprises two electrodes.

Clause 29. The system of clause 24 or 25, wherein the brain region is: hippocampus, septo-hippocampus, ACC, BLA, midline thalamus, insulate regions, medial septum, fimbria fornix, amygdala, or a combination thereof.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

It is proposed that a subject can be treated for suboptimal levels of empathy by modulating levels of type 2 theta oscillations in the subject's brain. Without intending to be limited by theory, it has been found that empathy in mice is correlated with observational fear. In particular, mice with higher observational fear also have higher levels of empathy to fellow mice, whereas mice with lower observational fear have lower levels of empathy to fellow mice. In addition, it has been found that type 2 theta oscillations are associated with greater empathy and greater observational fear. Therefore, increasing type 2 theta oscillations in a subject would increase empathy in the subject.

### Example 1—Experimental Methods

The experimental approaches used a combination of studying genetics and circuit mechanisms underlying theta oscillations and empathy, along with advanced ofMRI scans, computational modeling, and optogenetic functional ultrasound imaging.

A mice breeding program was conducted that resulted in a mice lineage with reduced observational fear, along with a second mice lineage with normal or increased levels of observational fear. The lineages were studied in order to uncover correlations between behaviors of empathy and observational fear with theta brain waves, genetics, optogenetics, ultrasound, fMRI, and ofMRI.

PLC-β1 mutant mice were used, which showed a severe deficit in observational fear learning in preliminary studies. The focus was on the rhythmic oscillation in the ACC and the amygdala. Here, the PLC-β1 function in the ACC excitatory neurons and synchronized 4-8 Hz theta oscillations in the ACC and BLA during observational fear was found to be involved in observational fear. These synchronized oscillations were type-2 hippocampal theta rhythms, and these rhythmic synchrony was shown to be selectively required for observational fear learning, but not for classical fear conditioning. Furthermore, the rise and fall of this rhythmic synchrony among the three brain regions (hippocampus, ACC, and BLA) temporally precede the on- and off-set of each freezing bout during observational fear. Finally, the strength of observational fear behavior could change bi-directionally by optogenetically changing the strength of the type-2 theta oscillations. Therefore, hippocampal type-2 theta rhythm synchronized in the long-range network consisting of the hippocampus, ACC, and amygdala drives the cognitive process underlying affective empathy leading to observational fear, without affecting conventional fear conditioning.

### Example 2—a PLC-β1 Deficiency in Pyramidal Neurons of the ACC Impairs Observational Fear and Excitability in Layer 2/3 Neurons

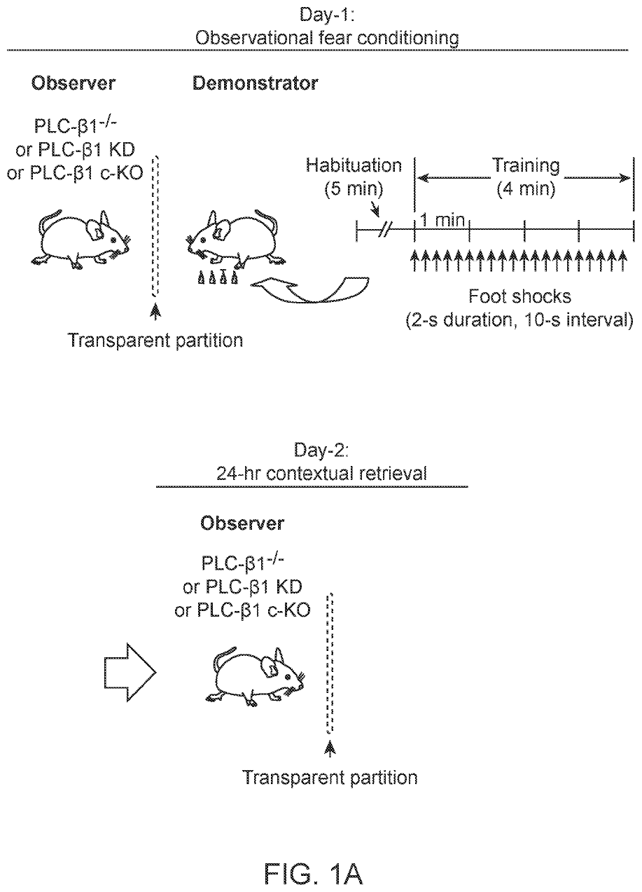

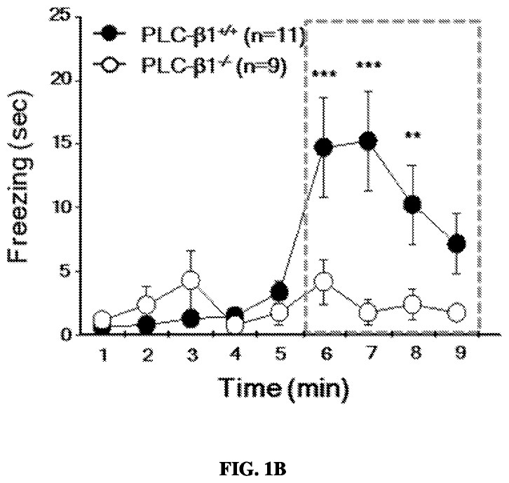

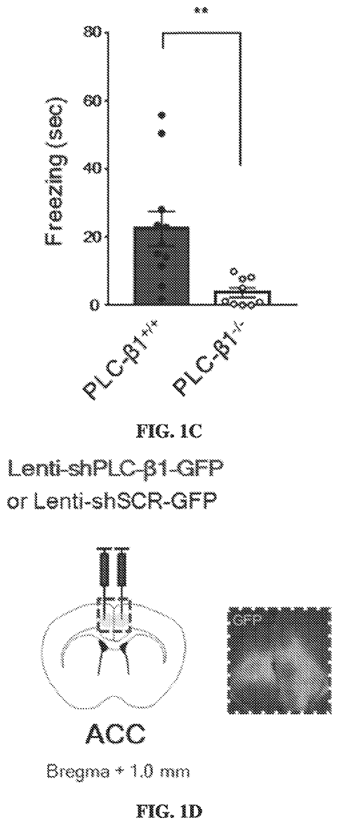

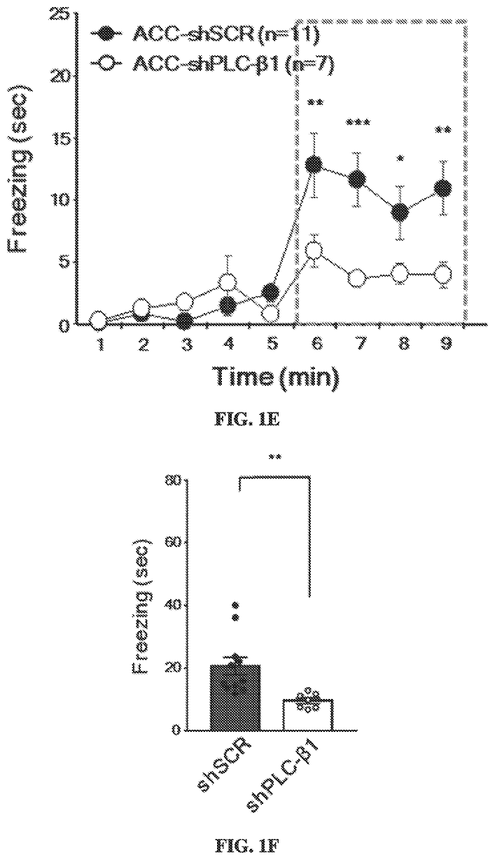

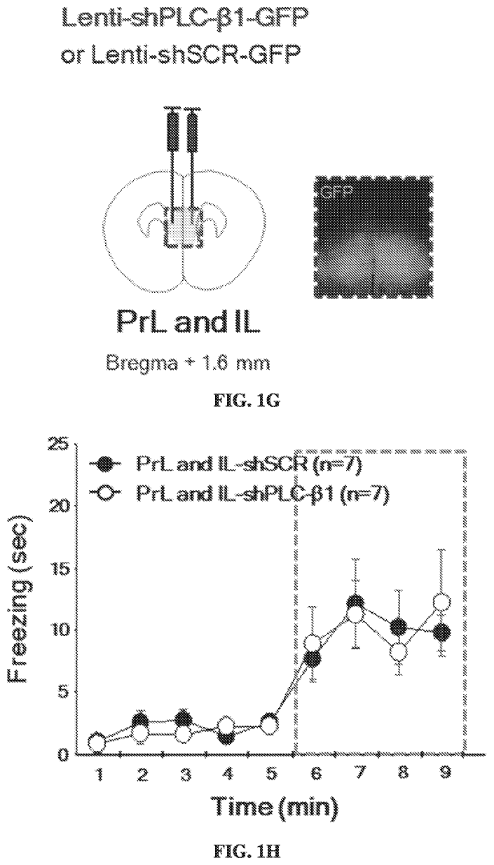

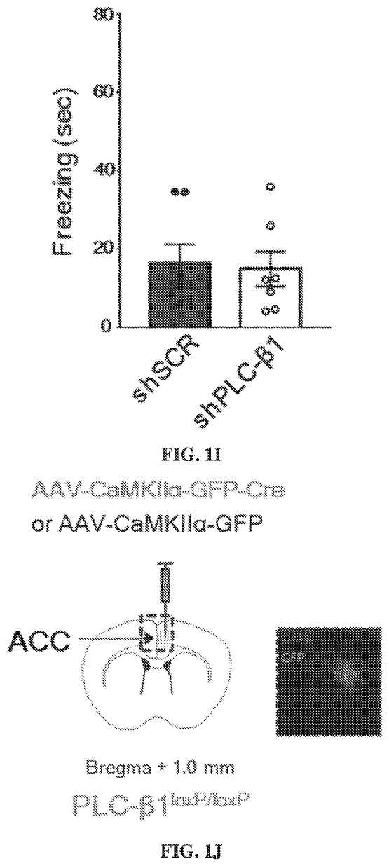

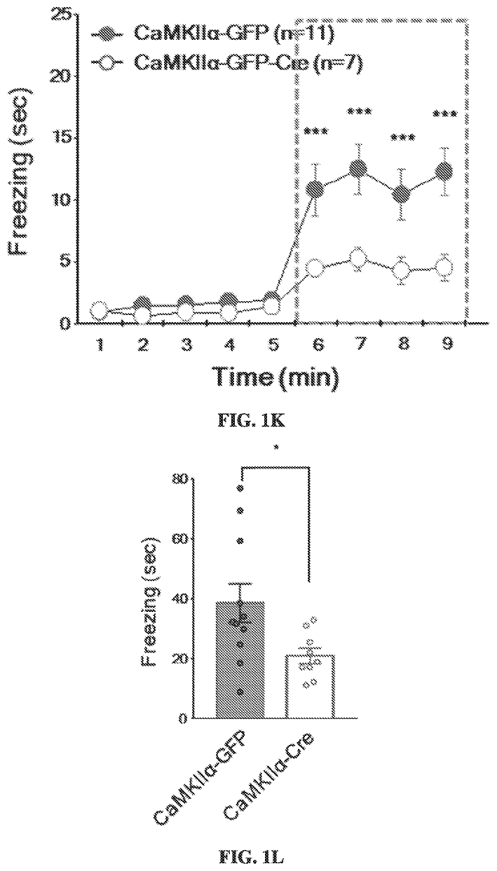

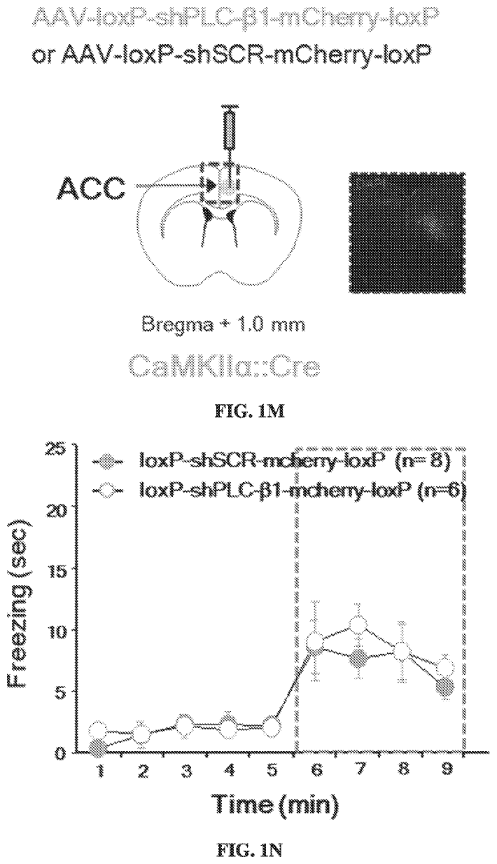

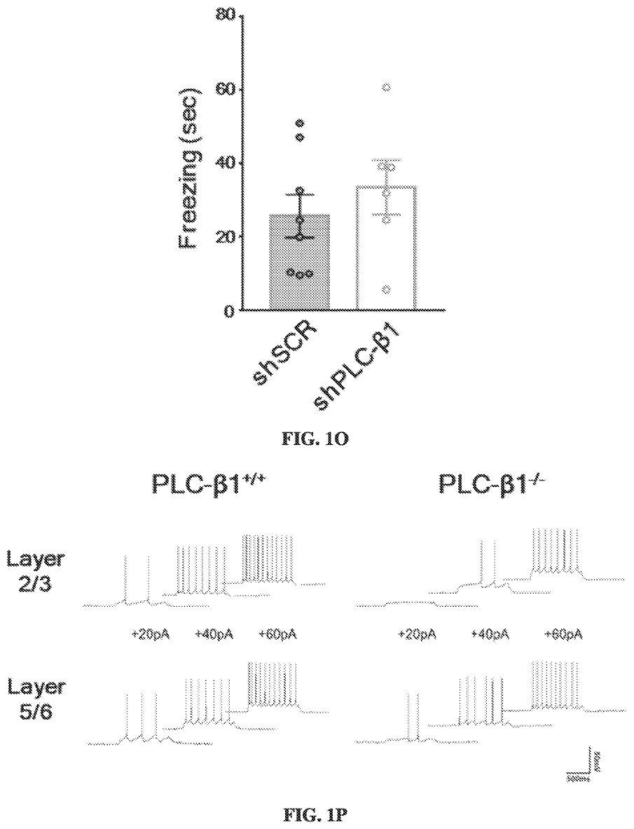

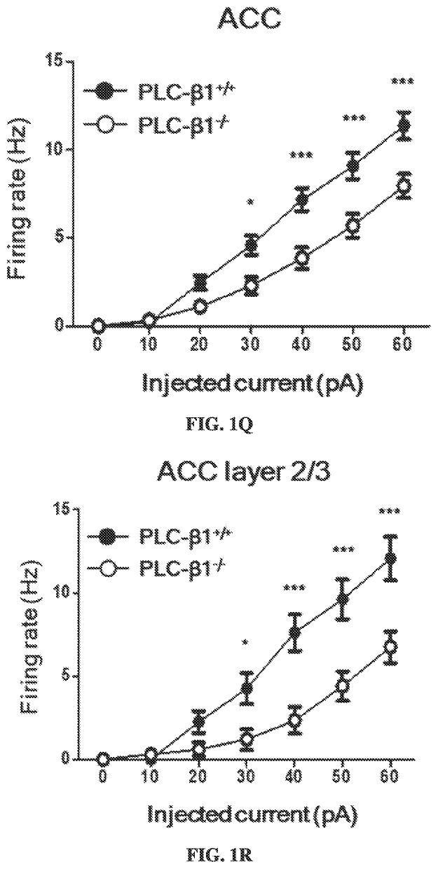

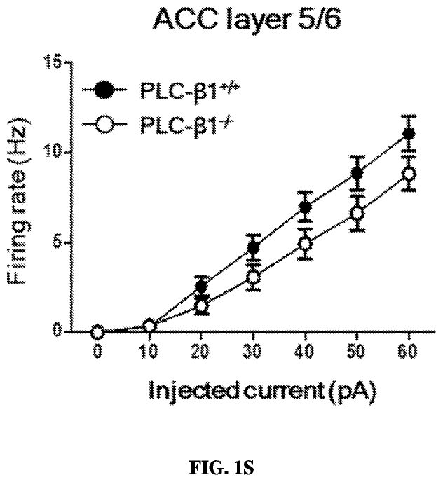

PLC-β1 mutant mice, a mouse model displaying multiple endo-phenotypes of schizophrenia including social deficits, show a deficit in observational fear learning. Therefore, systematic analysis of this mutant was performed at diverse levels, including behavior, cell, circuit, physiology and EEG analysis. To determine specifically whether PLC-β1 is involved in observational fear, observational fear learning assays were performed with PLC-β1 knockout (PLC-β1^{-/-}) mice using a previously described by Jeon et al. (2010) (FIG. 1A). Observer PLC-β1^{-/-} mice clearly showed decreased freezing behavior in this assay compared with wild-type observer mice (F_{1,18}=4.648, p<0.05; FIG. 1B). PLC-β1^{-/-} observer mice placed back in the same observing chamber, with the other chamber empty, 24-h after training displayed decreased freezing compared with wild-type observer mice (p<0.01, t(18)=3.213; FIG. 1C). Thus, PLC-β1 signaling is required for observational fear behavior in mice. PLC-β1 is abundantly expressed in regions of the medial prefrontal cortex (mPFC), including the ACC. For convenience, the ACC was distinguished from regions of the mPFC containing the prelimbic (PrL) and infralimbic cortex (IL). To determine whether the impaired observational fear in PLC-β1^{-/-} mice was attributable to a PLC-β1 defect in the ACC or the mPFC, a lentivirus encoding a small hairpin RNA (shRNA) targeting PLC-β1 mRNA (shPLC-β1) was bilaterally injected into the ACC (FIG. 1D) or the mPFC (FIG. 1G) of wild-type mice. During observational fear conditioning, observers administered an ACC-specific injection of shPLC-β1 exhibited significantly lower freezing levels than observers injected with the virus with a scrambled shRNA sequence (shSCR) (F_{1,16}=4.916, p<0.05; FIG. 1E). Contextual memory at 24-h was also decreased in observers administered an ACC-specific shPLC-β1 injection (p<0.01, t(16)=2.961; FIG. 1F). On the other hand, PLC-β1 knockdown in the mPFC did not affect observational fear conditioning (F_{1,12}=0.00406, P=0.950; FIG. 1H) and 24-h contextual memory (p=0.824, t(12)=0.228; FIG. 1I). In contrast to whole-body PLC-β1 knockout mouse, ACC-specific silencing of PLC-β1 did not affect contextual fear conditioning and 24-h context-dependent memory. This finding is consistent with previous reports that the ACC is involved in vicarious fear learning through observation, but not in classical conditioning that relies on direct experience of the noxious stimulus of foot shocks. Observer mice with ACC-specific PLC-β1 knockdown did not show changes in locomotion or anxiety level, as determined by the open-field test, light-dark transition test, and elevated plus-maze test. To assess the extent of gene silencing, mice were sacrificed after completion of behavioral tests and PLC-β1 expression was measured in ACC neurons immunohistochemically. PLC-β1-positive neurons in the ACC were markedly decreased in numbers in shPLC-β1-injected mice compared with shSCR-injected mice. Next, cell-types in the ACC of the mouse brain that expressed PLC-17 β1 were defined. Co-expression of PLC-β1 with calcium/calmodulin-dependent protein kinase II alpha (CaMKIIα) or glutamate decarboxylase 67 (GAD67) was examined by double-immunofluorescence analysis. PLC-β1-positive ACC neurons were mainly CaMKIIα labeled, with a lower frequency of GAD67-labeled cells. On average 94.91%±4.39% of CaMKIIα-labeled neurons and 32.72%±4.32% of GAD67-labeled neurons were also positive for PLC-β1. Thus, the majority of ACC excitatory neurons and some inhibitory neurons express PLC-1 β1.

To investigate the role of PLC-β1 in excitatory neurons of the ACC in observational fear, ACC excitatory neuron-specific PLC-β1 conditional knockout (PLC-β1ACC/Cre) mouse was generated using the Cre-LoxP recombination approach, by injecting AAV5-CaMKIIα-GFP-Cre into the ACC of PLC-β1loxP/loxP mice (FIG. 1J). PLC-β1ACC/Cre observer mice exhibited impaired observational fear (F_{1,18}=10.981, p<0.01; FIG. 1K) and 24-h contextual memory (p<0.05, t(18)=2.358; FIG. 1L) compared with control observers injected with AAV5-CaMKIIα-GFP (PLC-β1ACC/GFP). To determine the effect of PLC-β1 silencing in ACC non-excitatory neurons, including inhibitory neurons, on observational fear, AAV-loxP-U6-shPLC-β1-CMV-mCherry-loxP was delivered into the ACC of CaMKIIα-Cre mice (FIG. 11 1M). Knockdown of PLC-β1 in ACC non-excitatory neurons did not affect observational fear conditioning (F_{1,12}=0.162, p=0.695; FIG. 1N) and 24-h contextual memory (P=0.417, t(12)=−0.841; FIG. 1O).

After completion of behavioral tests, the mice were sacrificed and PLC-β1 expression in excitatory and inhibitory ACC neurons was measured employing double immunohistochemistry. PLC-β1 expression was decreased in ACC excitatory neurons of mice injected with AAV5-CaMKIIα-GFP-Cre compared with those injected with AAV5-CaMKIIα-GFP. Similarly, PLC-β1 expression was decreased in ACC inhibitory neurons of mice injected with AAV-loxP-U6-shPLC-β1-CMV-mCherry-loxP compared with those injected with AAV-loxP-20 U6-shSCR-CMV-mCherry-loxP.

To evaluate the cellular mechanisms underlying impaired observational fear in PLC-β1^{-/-} mice, the intrinsic firing properties were examined for ACC pyramidal neurons in brain slices using the patch-clamp recording technique. Activities of ACC pyramidal neurons in B6.129 PLC-β1^{+/+}/GAD65GFPtg and B6.129 PLC-β1^{-/-}GAD65GFPtg mice were compared by injecting depolarizing currents (10-pA increments, 7 steps, 1-s duration) into cells from a resting membrane potential adjusted to −60 mV (FIG. 1P). The frequency of action potentials evoked by depolarizing currents was significantly decreased in pyramidal neurons of PLC-β1^{-/-} mice compared with those of wild-type mice (F_{1,98}=10.86, p=0.0014, two-way repeated measures ANOVA; FIG. 1Q). Notably, this reduction in excitability was observed only in layer 2/3 ACC neurons (F_{1,34}=11.62, p=0.0017, two-way repeated measures ANOVA; FIG. 1R), but not in layer 5/6 neurons (F_{1,62}=2.74, p=0.1027, two-way repeated measures ANOVA; FIG. 1S). Taken together, these findings indicate that PLC-β1 signaling in excitatory, not inhibitory, neurons of the ACC is responsible for observational fear and excitability of layer 2/3 neurons in mice.

### Example 3—Reciprocal Circuits Between the ACC and the BLA are Necessary for Observational Fear but not for Classical Fear Conditioning

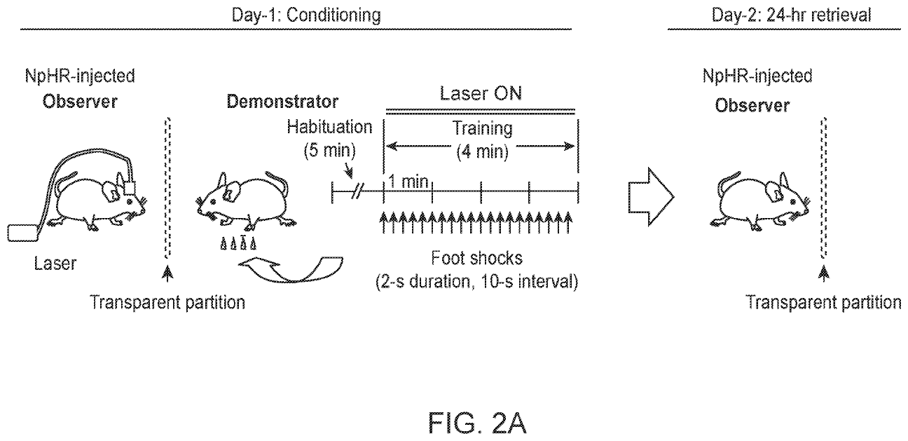

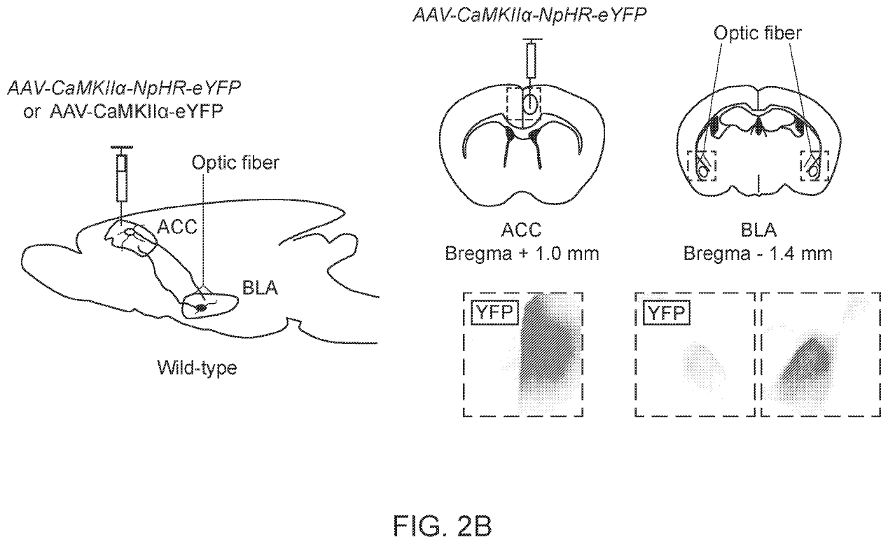

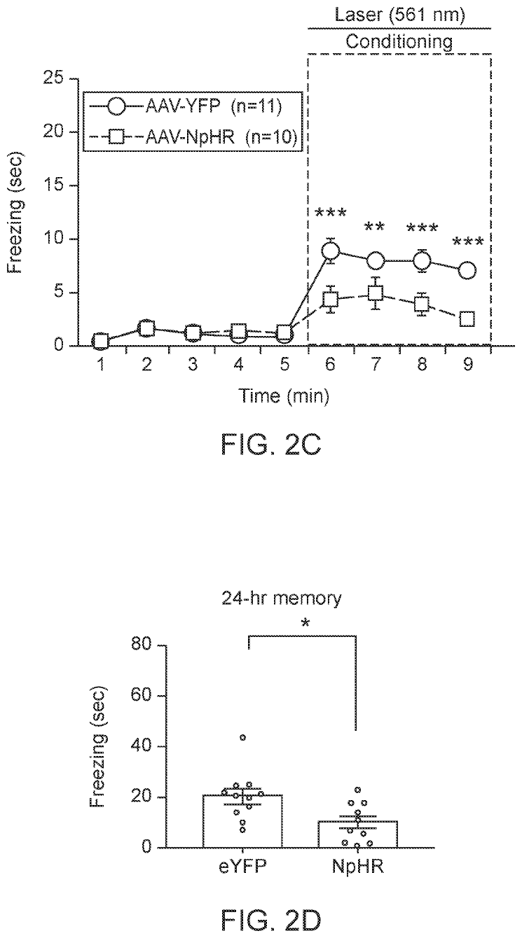

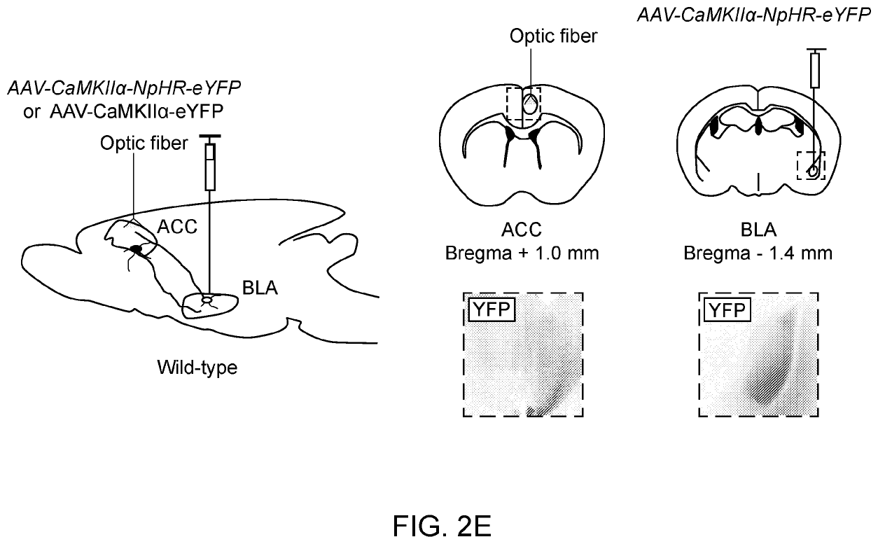

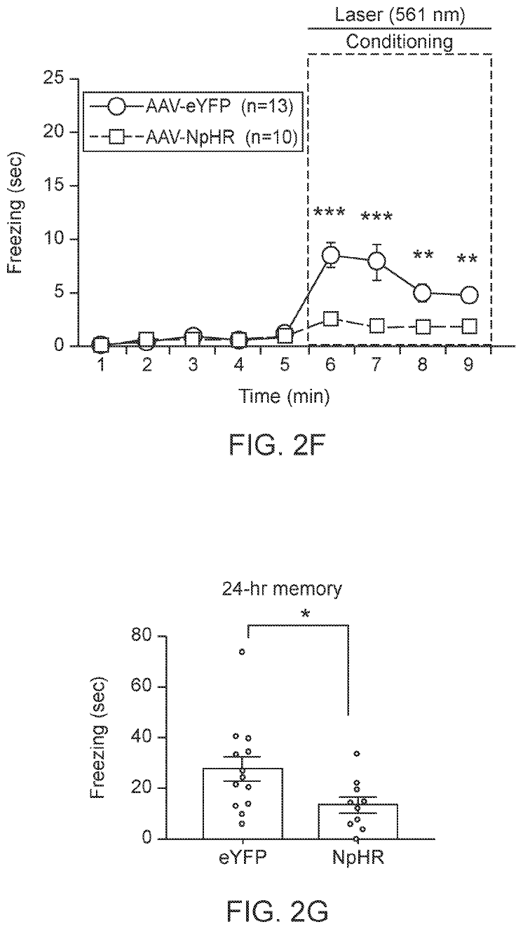

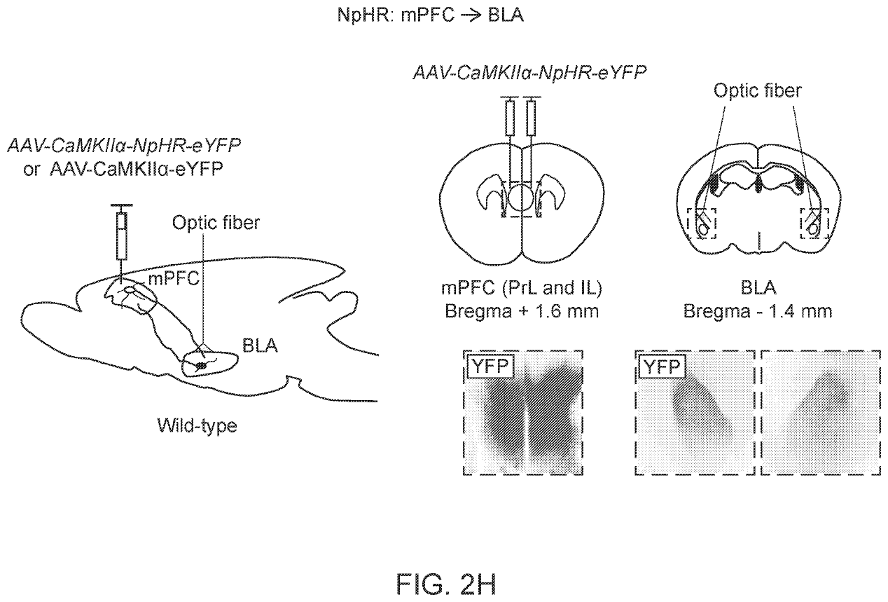

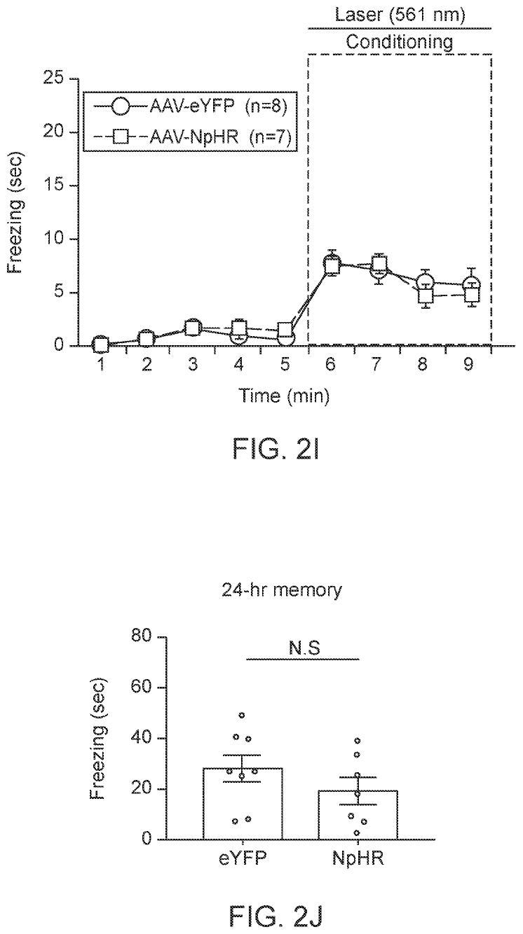

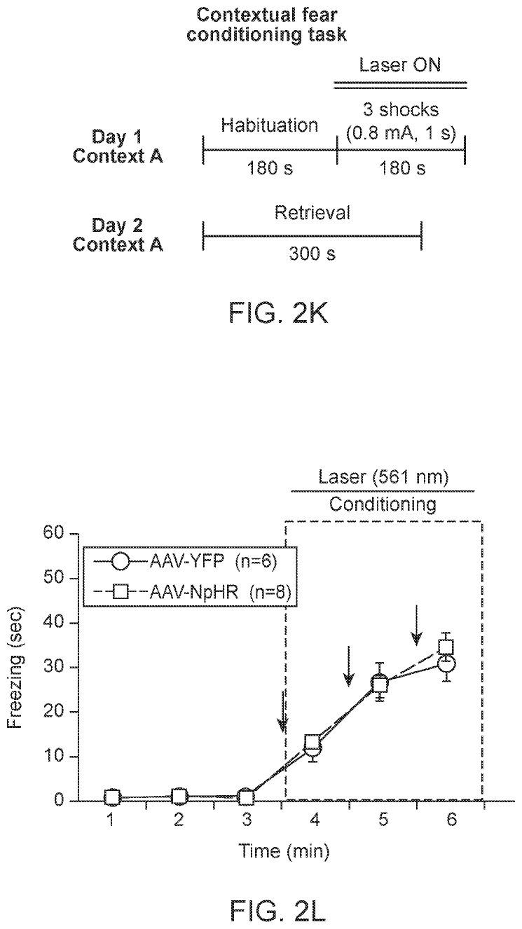

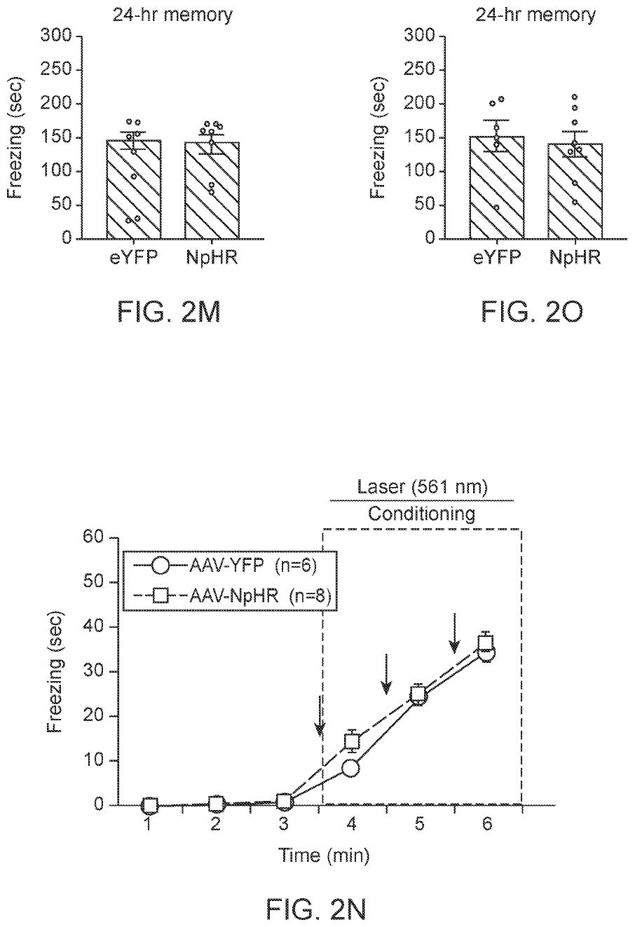

Previous anterograde and retrograde labeling studies have shown that layer 2/3 neurons, not layer 5/6 neurons, in the ACC and mPFC preferentially project to the BLA in mice. The ACC also receives amygdala inputs mainly from the BLA, demonstrating the reciprocity of ACC-BLA circuits. Studies using simultaneous recordings of local field potential (LFP) in the ACC and the lateral amygdala in freely behaving wild-type observer mice suggested a functional connectivity between the ACC and amygdala in observational fear. To investigate whether projections from the ACC or the mPFC to the BLA are necessary for observational fear, optogenetic approaches were used to inhibit the ACC-BLA or the mPFC-BLA circuit during observational fear (FIG. 2A). In these experiments, the ACC (FIG. 2B) or mPFC (FIG. 2H) was injected with AAV5-2 CaMKIIα-NpHR3.0-eYFP (NpHR) 6-wk before the observational fear task and the BLA was bilaterally illuminated with a yellow laser. Upon optogenetic silencing of axon terminals of ACC excitatory neurons within the bilateral BLA during observational fear conditioning, NpHR-injected mice showed reduced freezing compared with AAV5-CaMKIIα-eYFP (eYFP)-injected control mice (F_{1,19}=14.224, P=0.001; FIG. 2C). In contextual recall tests performed the next day, mice in the NpHR-injected group exhibited a significant reduction in freezing behavior compared with those in the eYFP-injected group (p<0.05, t(19)=−2.671; FIG. 2D). However, optogenetic inhibition of axon terminals of the mPFC, including PrL and IL excitatory neurons, within the bilateral BLA during observational fear did not affect conditioning (F_{1,13}=0.00232, p=0.962; FIG. 2I) and 24-h contextual memory retrieval (p=0.267, t(13)=1.160; FIG. 2J). Optogenetic inhibition of axon terminals of excitatory ACC neurons within the bilateral BLA (FIG. 2B) during classical contextual fear conditioning (FIG. 2K) also had no effect on training (F_{1,12}=0.0587, P=0.813; FIG. 2L) or 24-h contextual memory retrieval (p=0.787, t(12)=0.276; FIG. 2M).

To verify whether projections from the BLA to the ACC are involved in observational fear, excitatory projections from the BLA to the ACC were optogenetically inhibited during observational fear. To this end, the BLA was injected with NpHR and the ACC was illuminated with a yellow laser (FIG. 2E). Upon optogenetic silencing of excitatory BLA terminals within the ACC during observational fear conditioning (FIGS. 2A and 2E), NpHR-injected mice showed reduced freezing compared with eYFP-injected control mice (F_{1,21}=12.858, p<0.01; FIG. 2F). In contextual recall test performed the next day, mice in the NpHR-injected group exhibited reduced freezing behavior compared with mice in the eYFP-injected group (p<0.05, t(21)=−2.251; FIG. 2G). On the other hand, optogenetic inhibition of axon terminals of excitatory BLA neurons within the ACC (FIG. 2E) during contextual fear conditioning (FIG. 2K) had no effect on training (F_{1, 12}=1.398, p=0.260; FIG. 2N) or 24-h contextual memory retrieval (p=0.698, t(12)=0.398; FIG. 2O). Collectively, these findings suggest that reciprocal excitatory circuits between the ACC and the BLA are necessary for observational fear, but not classical contextual fear conditioning.

### Example 4—the 4-8 Hz Oscillation in the ACC-BLA Circuit is Absent in the ACC-Specific PLC-β1 Knock-Down Mice which are Impaired in Observational Fear

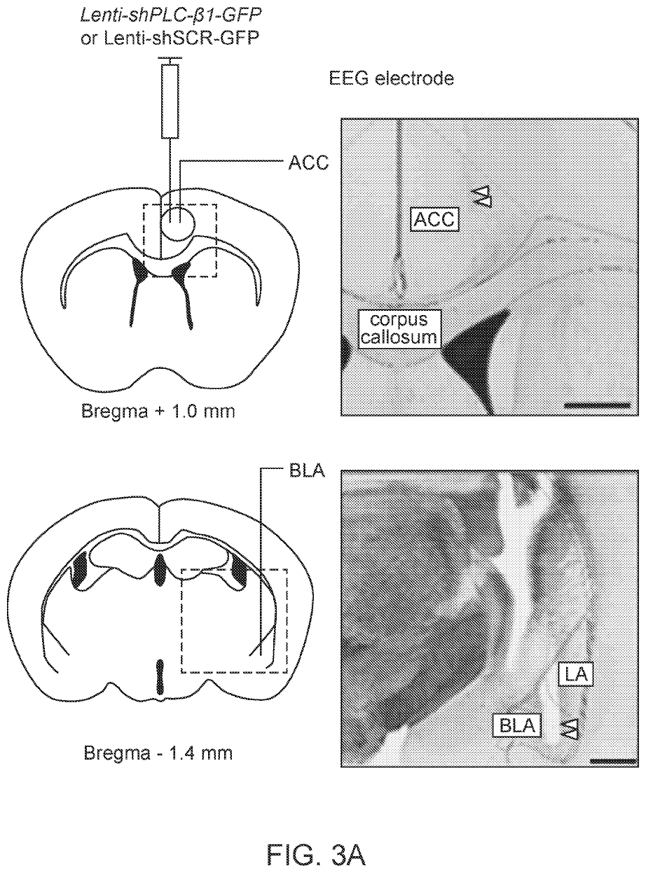

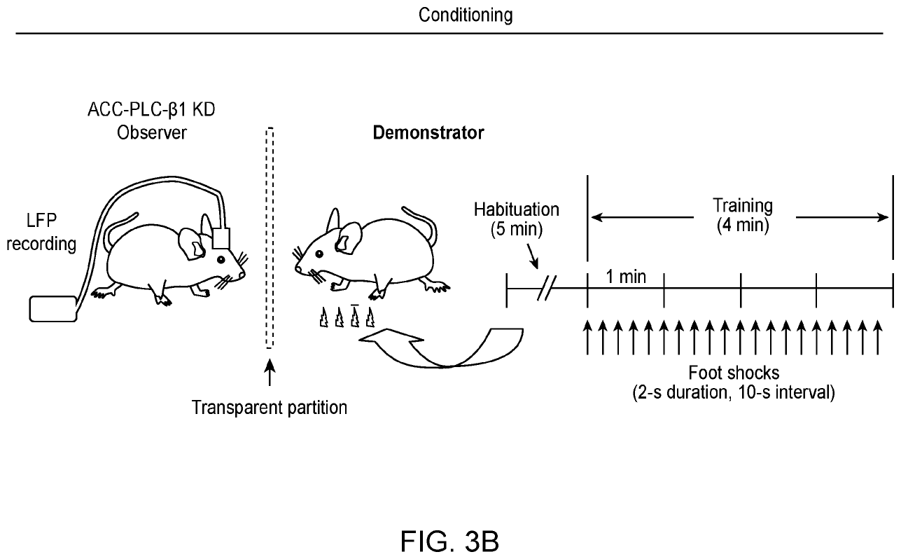

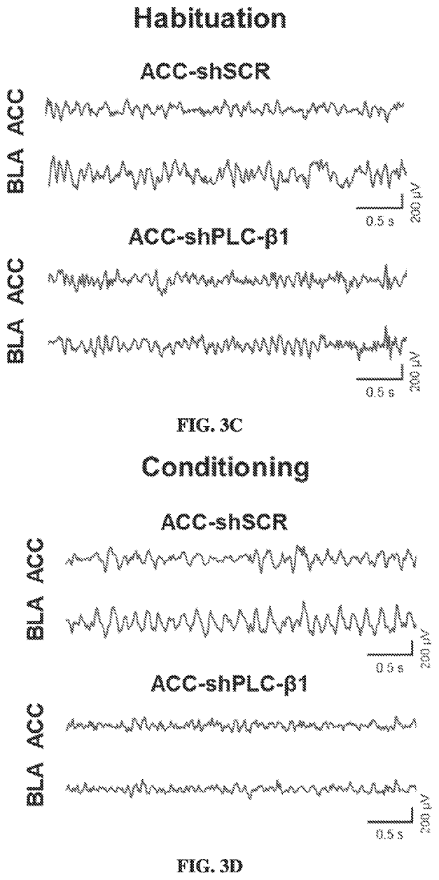

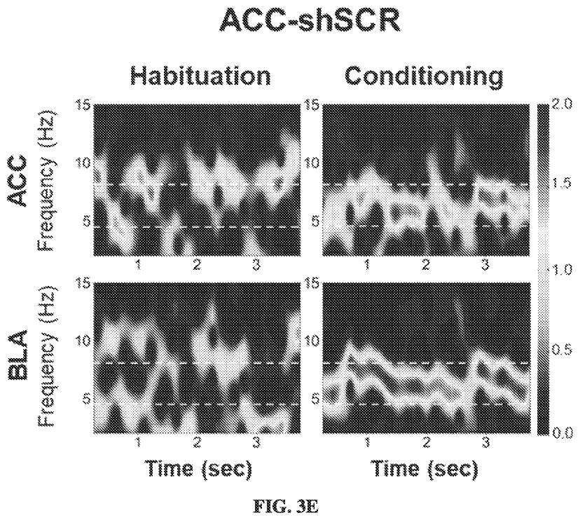

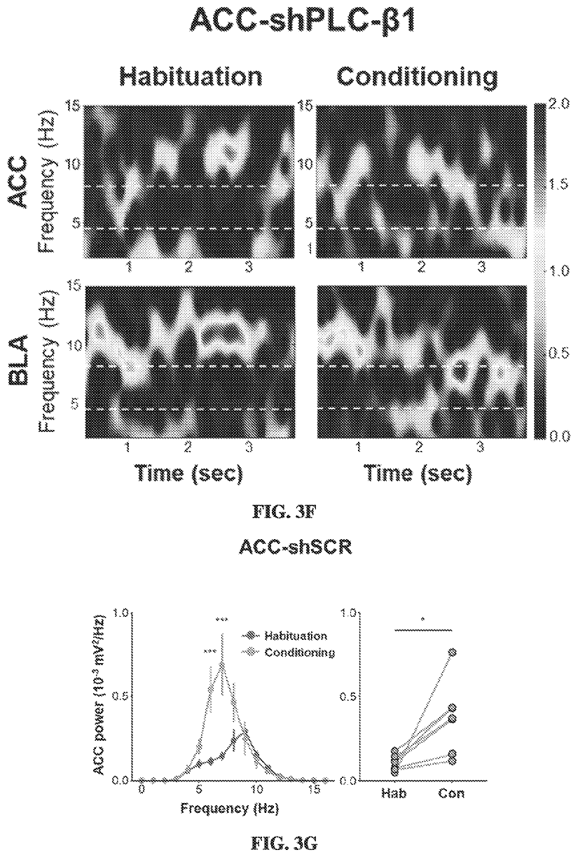

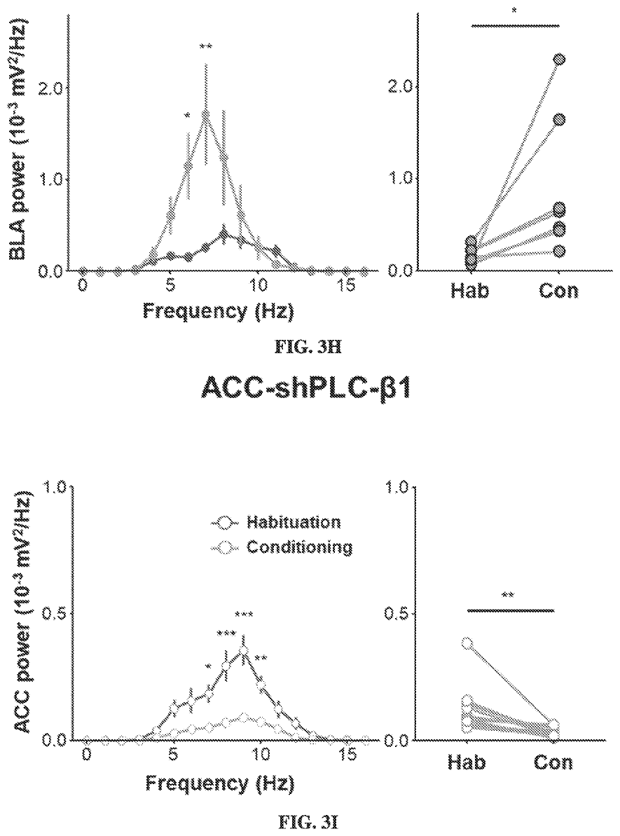

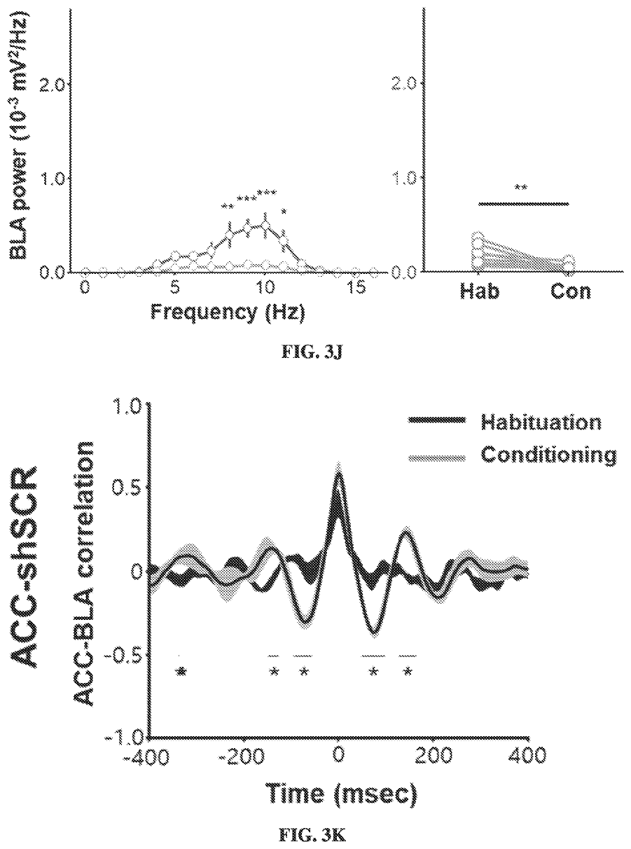

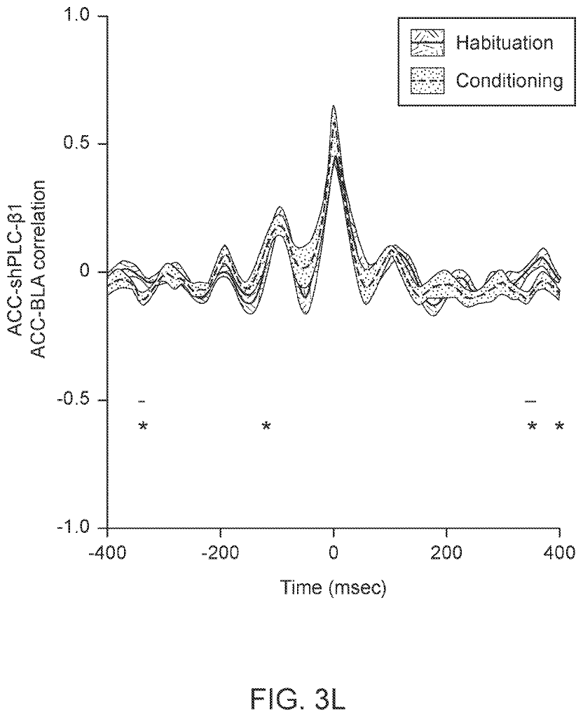

The emergence of 4-8 Hz synchronized oscillations between the ACC and BLA during observational fear was previously shown. Thus, whether these synchronized oscillations are absent in ACC-PLC-Pβ1-knockdown mice that are impaired in observational fear, following the procedures as described in Methods was teste. To this end, LFP recordings were performed in the ACC and BLA during observational fear conditioning in observer mice with ACC-specific injection of either shPLC-β1 or shSCR control virus (FIG. 3A). LFP recordings obtained during the habituation and inter-shock periods of the conditioning were used to estimate power spectrum density and cross-correlograms for each mouse (FIGS. 3B-D). First, in the control mice, the emergence of 4-8 Hz synchronized oscillations between the ACC and BLA during observational fear was confirmed. Thus, a significant modulation was found in the LFP theta power from habituation to conditioning in shSCR-injected mice in the ACC (ACC interaction condition x frequency: F_{8, 96}=6.155, p<0.001, two-way repeated measures ANOVA followed by Bonferroni post hoc test; 4-8 Hz, habituation to conditioning: p<0.05, t(6)=3.2792, paired t-test; FIGS. 3E and 3G), and in the BLA (BLA interaction condition x frequency: F_{8, 96}=5.697, p<0.001, two-way repeated measures ANOVA followed by Bonferroni post hoc test; 4-8 Hz habituation to conditioning: p<0.05, t(6)=2.5972, paired t-test; FIGS. 3E and 3H). In a striking contrast to the control group, shPLC-β1-injected mice showed a significant decrease in the power of the LFP spectra at 4-8 Hz from habituation to conditioning. (ACC interaction condition x frequency: F_{8,144}=4.401, p<0.001, two-way repeated measures ANOVA followed by Bonferroni post hoc test; 4-8 Hz habituation to conditioning: p<0.01, t(9)=−3.2894, paired t-test; FIGS. 3F and 3I) (BLA interaction condition x frequency: F_{8,144}=4.016, p<0.001, two-way repeated measures ANOVA followed by Bonferroni post hoc test; habituation to conditioning: p<0.01, t(9)=−3.3201, paired t-test; FIGS. 3F and 3J).

In addition to the decreased theta power, synchronization between the ACC and BLA was also impaired in shPLC-β1-injected observer mice during observational fear. The cross-correlation analysis in the control, shSCR-injected, mice showed a significantly increased correlation during the conditioning phase, especially at the second peak corresponding to the theta frequency (average distance 0 lag peak to the second peak: 142 ms [7.046 Hz]; FIG. 3K, light-blue trace), again confirming previous observation of theta synchrony between the ACC and BLA. On the other hand, a similar cross-correlation analysis in the shPLC-β1-injected mice did not show any sign of synchrony during observational fear (FIG. 3L). These results suggest that disruption of theta synchronization in the ACC and BLA is linked to the impaired observational fear resulting from the PLC-β1 deletion in the ACC. These findings indicate that PLC-β1 in the ACC is required for synchronized 4-8 Hz oscillations in the reciprocal circuit between the ACC and the BLA, and for observational fear.

### Example 5—Type-2 Theta Synchrony in the Long-Range Network Consisting of the Hippocampus, ACC, and Amygdala is Essential for the Expression of Observational Fear

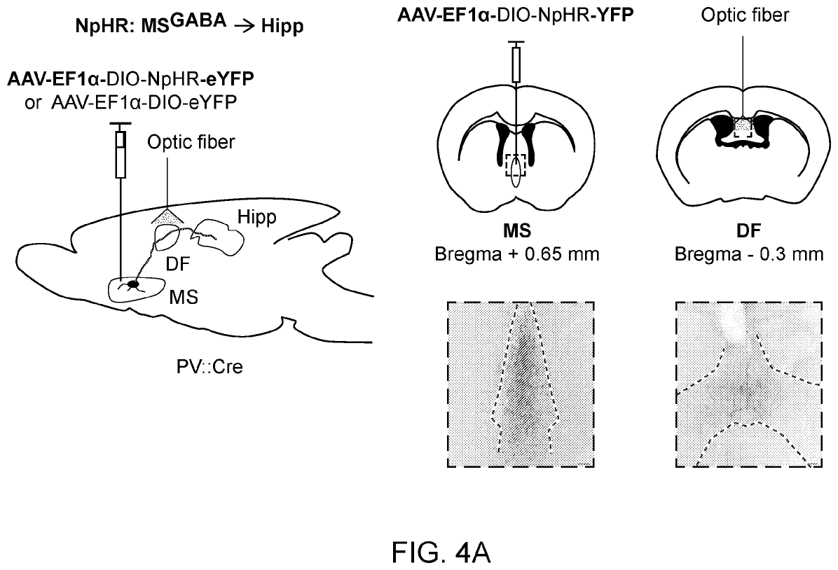

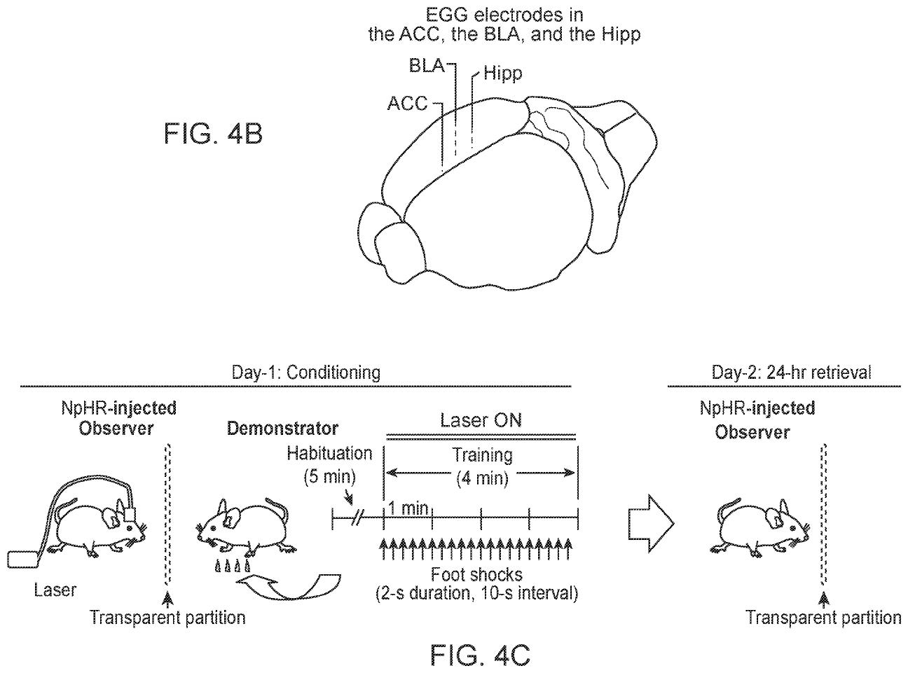

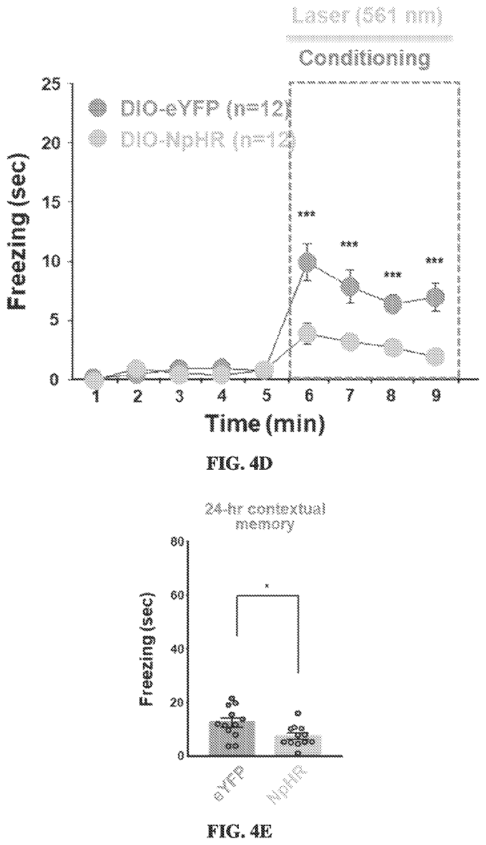

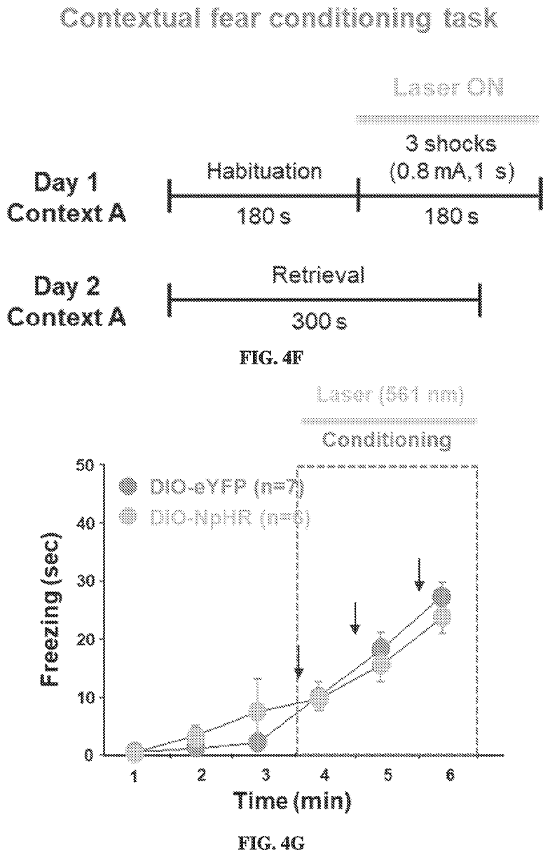

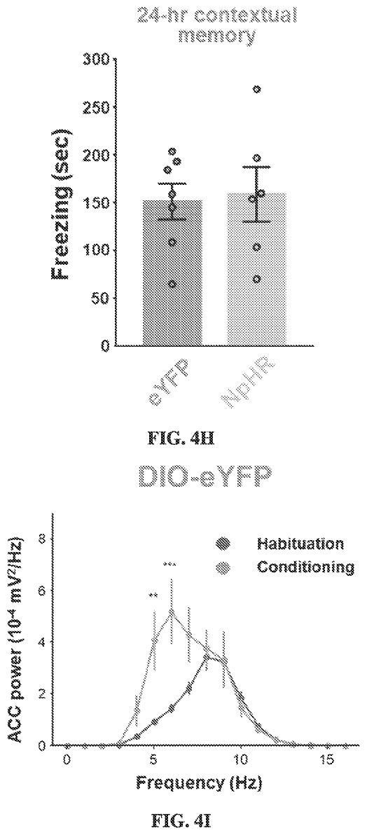

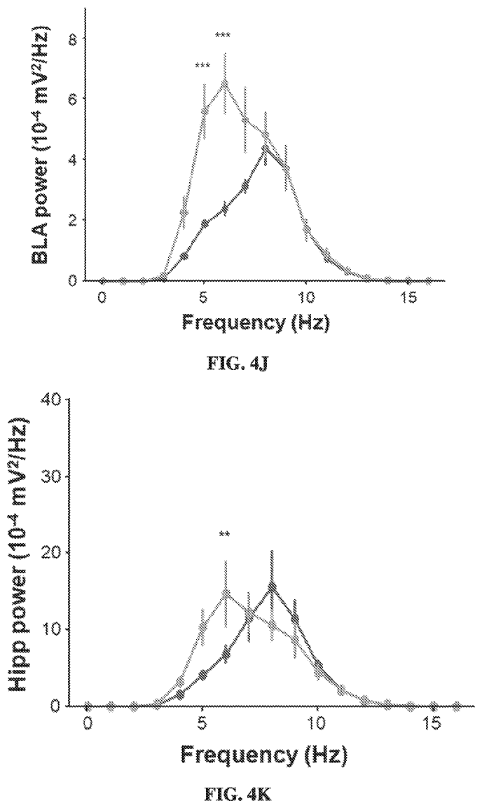

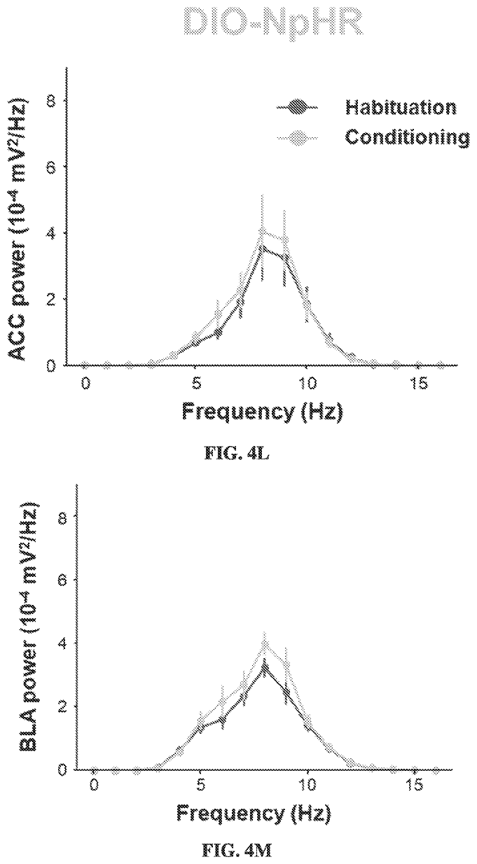

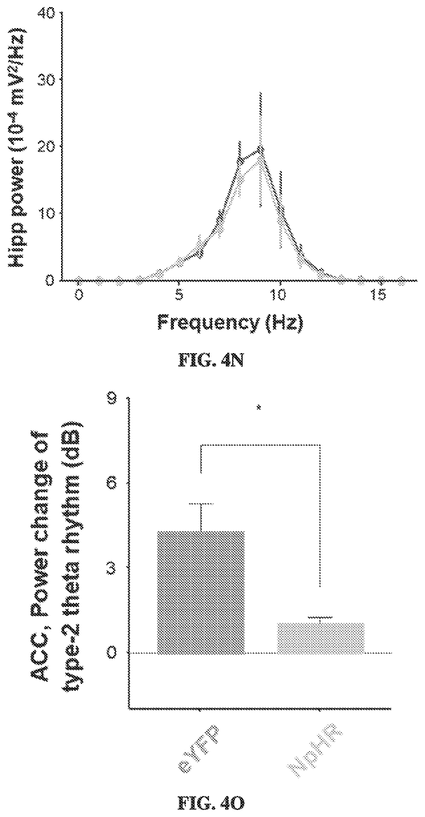

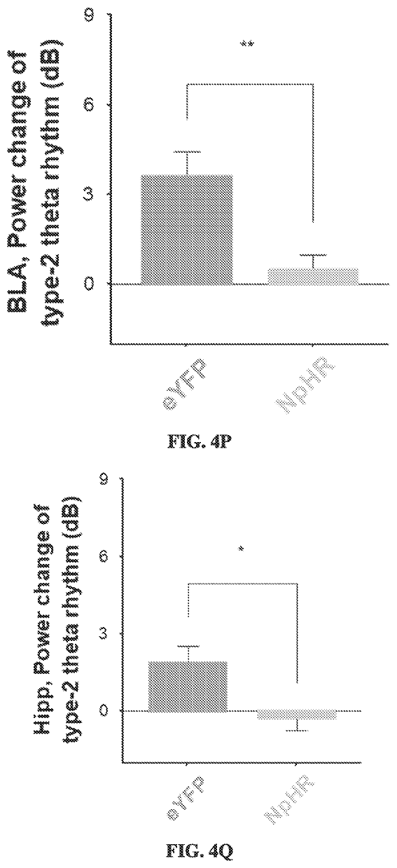

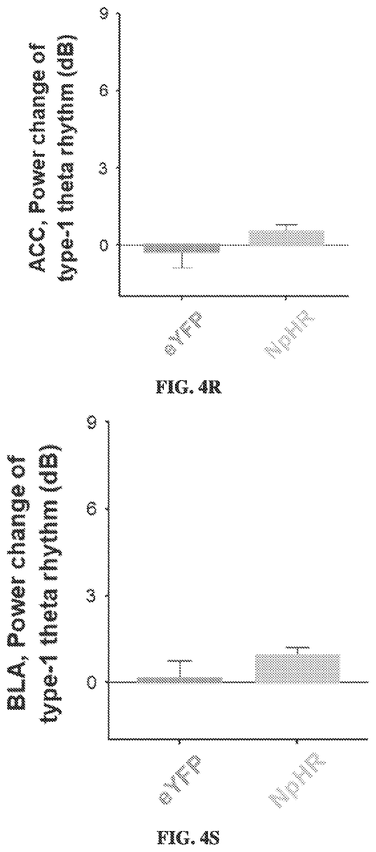

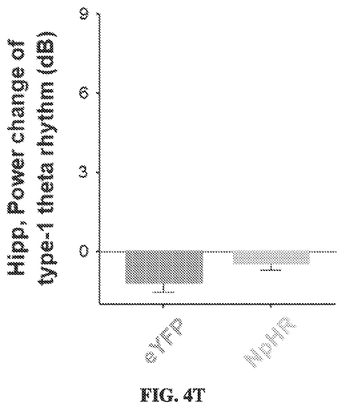

To determine whether this synchronized 4-8 Hz rhythm in the ACC and BLA is in fact hippocampal theta rhythm, type-2 hippocampal theta rhythm was selectively modulated using the method previously described by Gangadharan et al. Inhibiting GABAergic projections from the medial septum (MS) to the hippocampus is known to specifically reduce type-2, but not type-1, hippocampal theta rhythm in mice. To this end, the MS of PV-Cre transgenic mice was injected with AAV5-EF1α-DIO-NpHR3.0-eYFP (DIO-NpHR) 4-weeks before the observational fear assay. Then, the dorsal fornix was illuminated by a yellow laser during observational fear learning (FIG. 4A). Such light illuminations are expected to selectively affect the septal GABAergic projections to the hippocampus and thus attenuate type-2 theta selectively.

First, whether such treatment will affect observation fear behavior was examined Upon optogenetic inactivation of septo-hippocampal GABAergic fibers within the dorsal fornix during observational fear conditioning (FIG. 4C), DIO-NpHR-injected mice showed reduced freezing compared with the mice injected with the control, AAV5-EF1α-DIO-eYFP (DIO-eYFP) virus (F_{1, 22}=17.277, p<0.001; FIG. 4D). In contextual recall tests performed the next day, mice in the DIO-NpHR-injected group exhibited significantly reduced freezing behavior compared with mice in the DIO-eYFP-injected group (p<0.05, t(22)=2.480; FIG. 4E). In contrast, NpHR-mediated inhibition of septo-hippocampal GABAergic fibers during classical contextual fear conditioning (FIG. 4F) had no effect on training (F_{1, 11}=0.00289, p=0.958; FIG. 4G) and 24-h contextual memory retrieval (p=0.817, t(11)=0.238; FIG. 4H), indicating that the type-2 hippocampal rhythms are selectively required for observational fear, but not for fear in general.

The role of type-2 hippocampal theta rhythm in observational fear was also confirmed using another approach. MS-selective silencing of PLC-β4 is known to attenuate type-2 theta rhythm in the hippocampus of mice. Accordingly, MS-specific PLC-β4-knockdown mice were generated using shRNA targeting PLC-β4 (shPLC-β4) and then subjected these mice to the observational fear task. MS-8 restricted PLC-β4-knockdown observer mice exhibited impaired observational fear (F_{1,21}=9 7.423, p<0.05) and 24-h contextual memory (p<0.05, t(21)=−2.545) compared with control shRNA (shControl)-injected observers. Similar to the optogenetic experiments described above, there was no difference between shPLC-β4- and shControl-injected groups in classical contextual fear conditioning (F_{1,9}=4.847, p=0.055) and 24-h contextual fear memory (p=0.961, t(9)=0.0499).

Next, to confirm whether 4-8 Hz activities within the ACC and BLA is in fact affected by optogenetic inhibition of hippocampus-projecting GABAergic neurons in the MS during observational fear, LFP was measured in the ACC, BLA, and hippocampus during observational fear conditioning in the same observer mice with an MS-specific injection of DIO-NpHR or DIO-eYFP (FIG. 4B). Optogenetic silencing did not significantly change the occurrence and detection of overall 4-12 Hz oscillations. Comparative analysis of the power between the 4-8 Hz and 8-12 Hz frequency, however, revealed that in the control, DIO-eYFP-injected, observers the LFP power was significantly increased specifically in the 4-8 Hz frequency, but not in the 8-12 Hz, from habituation to conditioning in the three brain regions. The ACC (ACC interaction condition x frequency: F_{8,1}96=4.743, P<0.001, two-way repeated measures ANOVA followed by Bonferroni post hoc test; FIG. 4I), the BLA (BLA interaction condition x frequency: F_{8, 96}=6.248, p<0.001, two-way repeated measures ANOVA followed by Bonferroni post hoc test; FIG. 4J), and the hippocampus (hippocampus interaction condition x frequency: F_{8, 96}=3.061, p<0.01, two-way repeated measures ANOVA followed by Bonferroni post hoc test; FIG. 4K). In contrast, DIO-NpHR-mediated optogenetic inhibition of the MS GABAergic projections to the hippocampus abrogated such modulations in the LFP power from habituation to conditioning in the three regions. The ACC (ACC interaction condition x frequency: F_{8, 80}=0.188, p=0.992, two-way repeated measures ANOVA followed 10 by Bonferroni post hoc test; FIG. 4L), the BLA (BLA interaction condition x frequency: F_{8, 80}=0.743, p=0.653, two-way repeated measures ANOVA; FIG. 4M), and the hippocampus (hippocampus interaction condition x frequency: F_{8, 80}=0.081, p=1.000, two-way repeated measures ANOVA; FIG. 4N). These results demonstrate that suppression of hippocampal type-2 theta in fact interferes with observational fear behavior.

Importantly, the modulation in hippocampal theta oscillations during observational fear was specific to type-2 theta (FIG. 4K), and silencing of septo-hippocampal GABAergic projections selectively blocked this modulation in type-2 theta oscillations (P<0.05, t(11)=−2.4869; FIG. 4Q) without affecting 8-12 Hz type-1 theta oscillations (p=0.1444, t(11)=1.5716; FIG. 4T). Consistently with the type-2-specific block of hippocampal theta oscillations, a similar block in 4-8 Hz LFP power was observed in the ACC (P<0.05, t(11)=−2.9678; FIG. 4O) and the BLA (P<0.01, t(11)=−3.1829; FIG. 4P). On the other hand, changes in the power of the 8-12 Hz theta rhythm in the ACC and BLA were not different between DIO-eYFP- and DIO-NpHR-injected mice (ACC: p=0.3189, t(11)=1.0439; BLA: p=0.3061, t(11)=1.0734; FIGS. 4R and 4S).

Optogenetic suppression of type-2 hippocampal theta oscillations also blocked synchronization between these three brain areas. DIO-eYFP-injected control observers showed an increased correlation in the 4-8 Hz, type-2 theta range among the three brain regions (average distance 0 lag peak to second peak: ACC-BLA, 134 ms [7.46 Hz]; ACC-hippocampus, 163 ms [6.13 Hz]; BLA-hippocampus, 175 ms [5.71 Hz]). In contrast, such modulation in type-2 theta synchrony from habituation to conditioning was abrogated in DIO-NpHR-injected observers. Non zero-lag phase differences between brain regions suggest that the synchronization during observational fear is not due to volume conduction in our recordings.

Taken together, these findings show that the type-2 theta rhythm synchrony in the long-range network of the hippocampal-cingular-amygdalar circuit is essential for the expression of observational fear.

### Example 6—the Power Modulation in Type-2 Theta is Temporally Coupled with the Freezing Behavior, and the Degree of this Power Modulation Predicts the Magnitude of Observational Fear

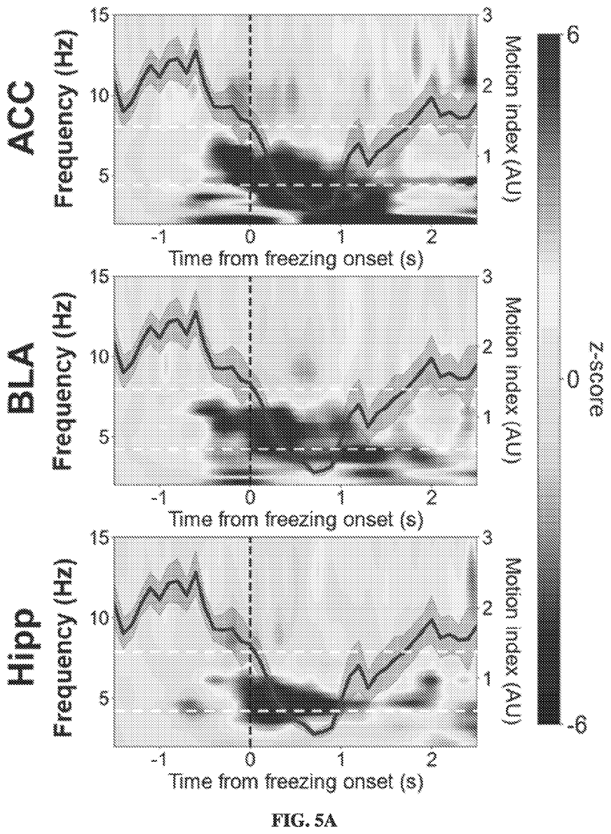

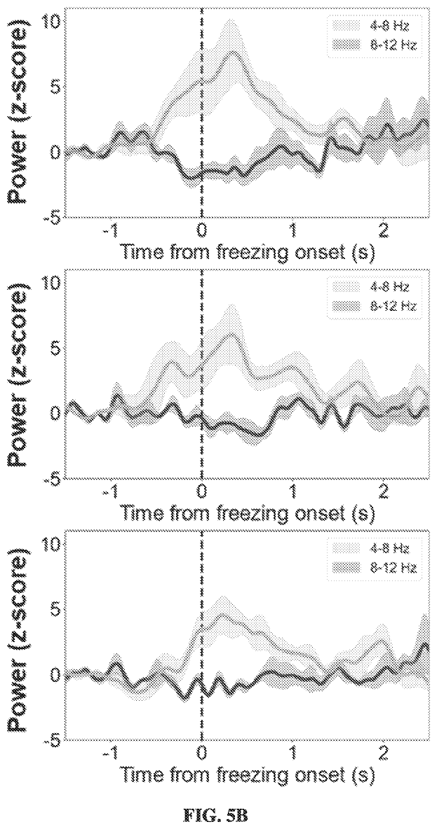

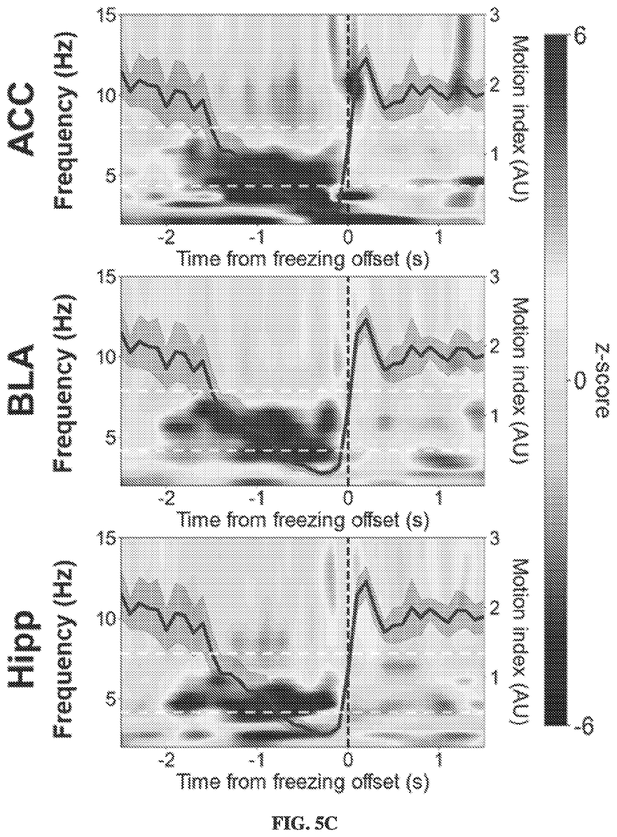

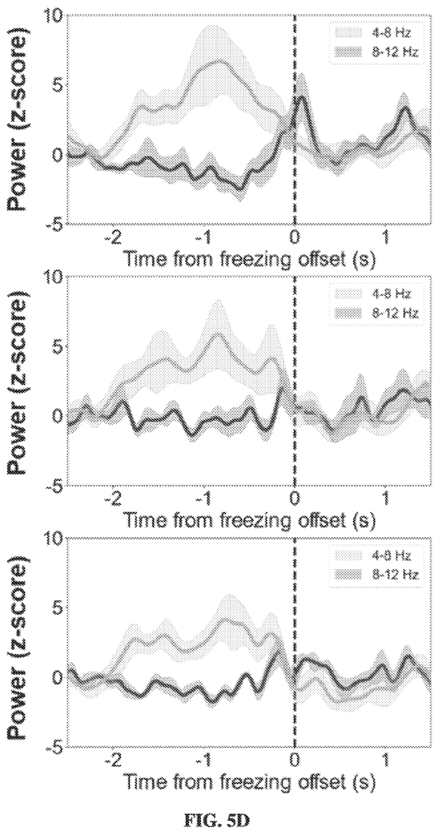

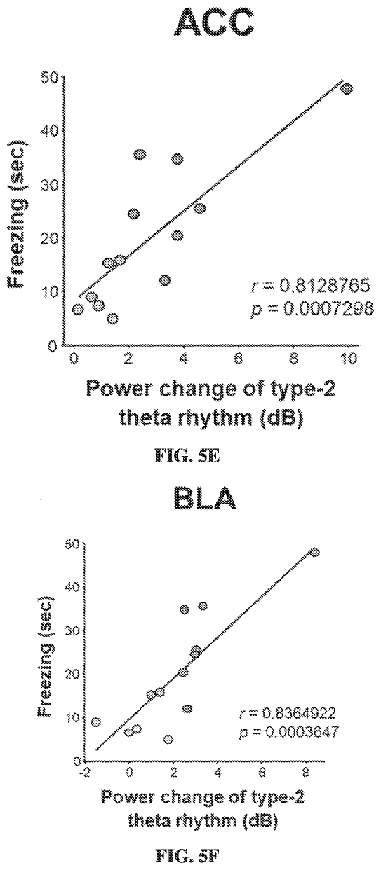

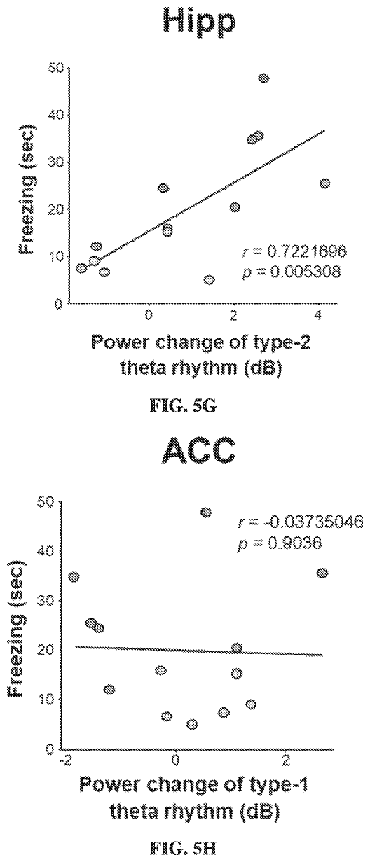

To examine the temporal relationship between type-2 hippocampal theta rhythm and the freezing bouts during observational fear, the temporal progression of type-2 hippocampal theta rhythm around the onset and offset of freezing in the control, DIO-eYFP-22 injected, observers was analyzed (FIGS. 5A-D). These experiments showed that the power of type-2 theta rhythms in the hippocampus-cingulate-amygdala circuit were increased immediately before the onset of freezing (FIGS. 5A and 5B), sustained during freezing, and then terminated just prior to the offset of freezing (FIGS. 5C and 5D), revealing a tight temporal coupling between the type-2 theta modulation and the freezing behavior.

Next, whether the strength of the type-2 theta is related to the magnitude of observational fear behavior was examined. The degree of power modulation in LFP at 4-8 Hz from habituation to conditioning was also positively correlated with the freezing level in the mouse (Pearson's correlation analysis: ACC, r=0.813, p<0.001; BLA, r=0.836, P<0.001; hippocampus, r=0.722, p<0.01; FIGS. 5E-G). On the other hand, there was no significant correlation between the change in the level of type-1 theta rhythm and the freezing level (FIGS. 5H-J), although type-1 rhythm seems to be decreasing moderately during the freezing (FIGS. 5A-D).

In summary, the modulation of type-2 theta rhythms in these three brain regions are tightly time-locked with and quantitatively correlated with freezing behavior during observational fear.

### Example 7—Upregulation of the Type-2 Hippocampal Theta Rhythm Modulations Observational Fear

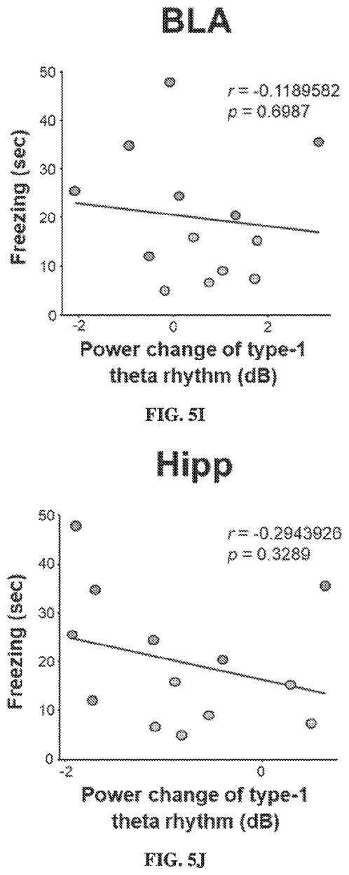

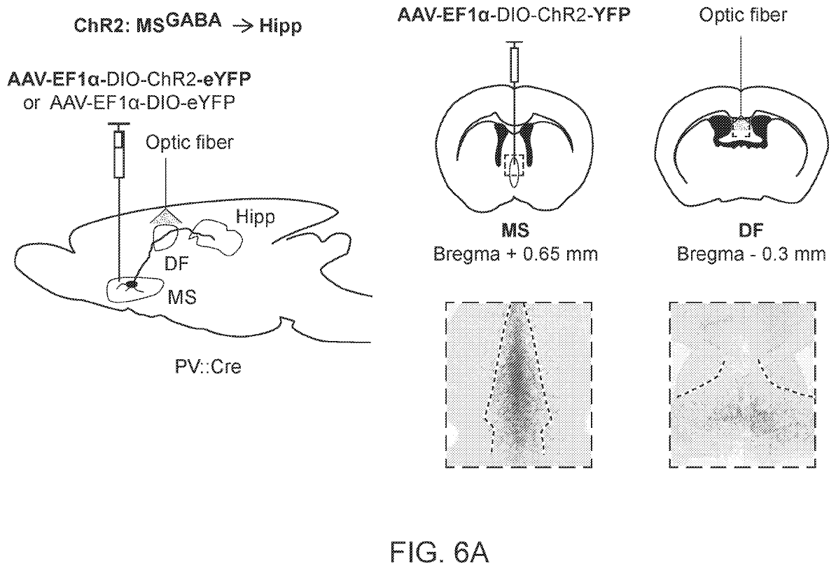

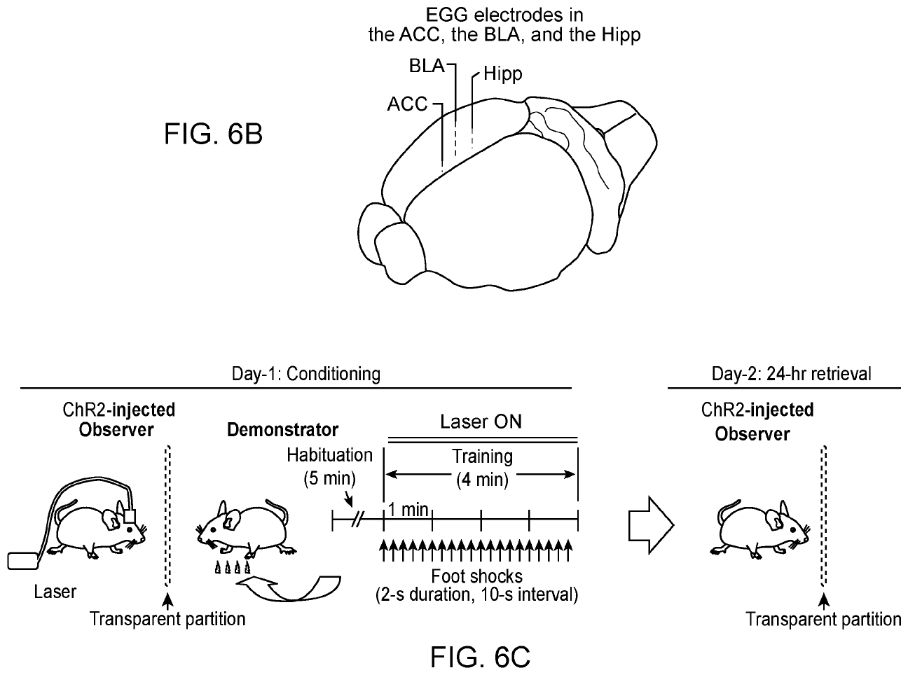

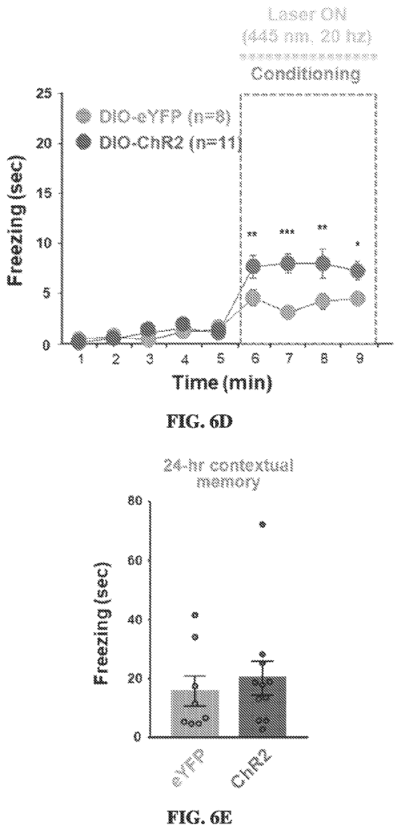

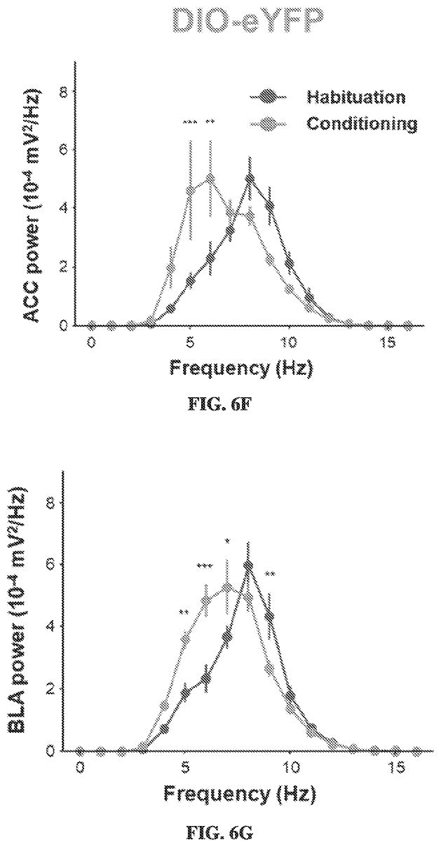

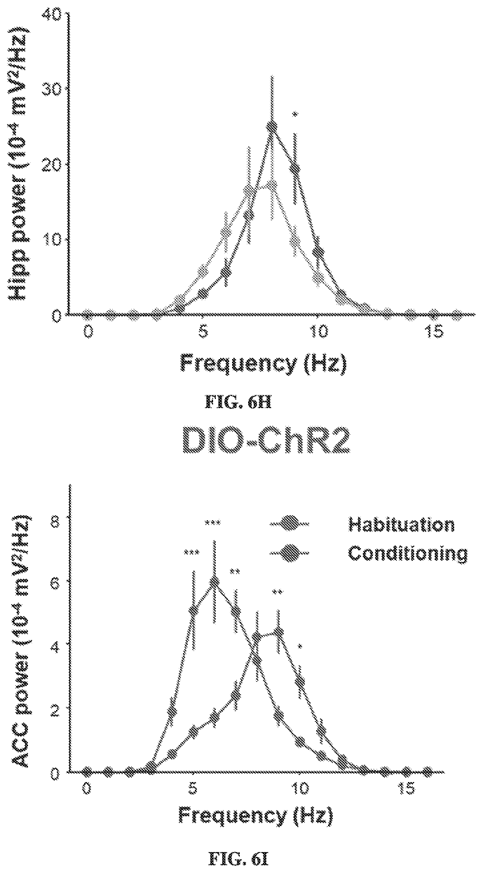

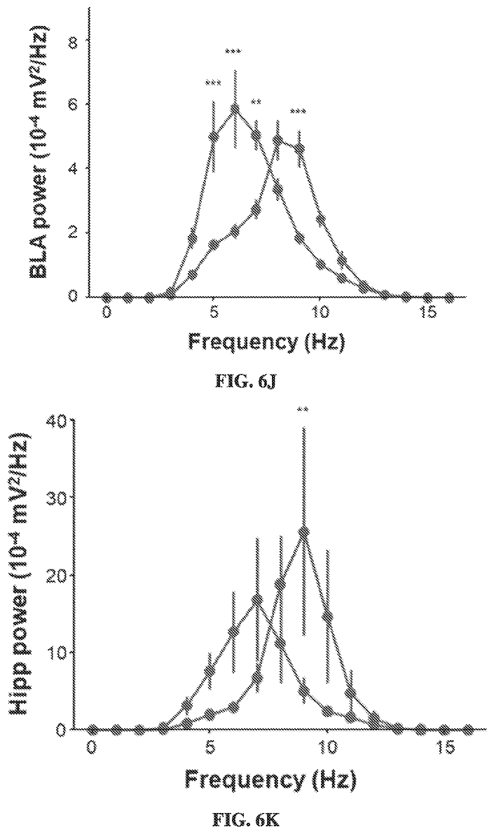

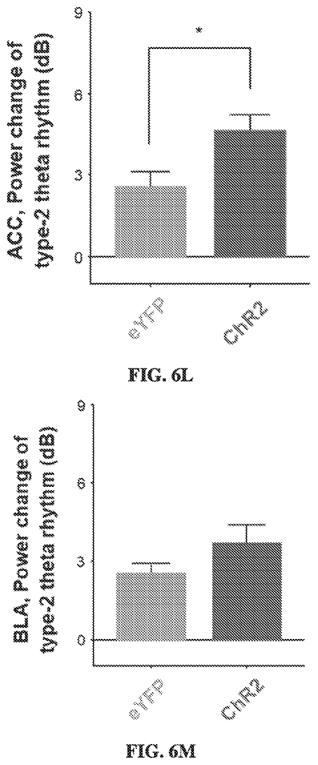

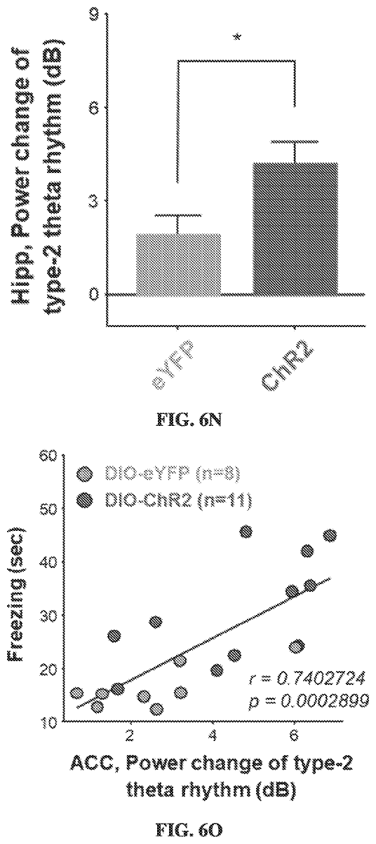

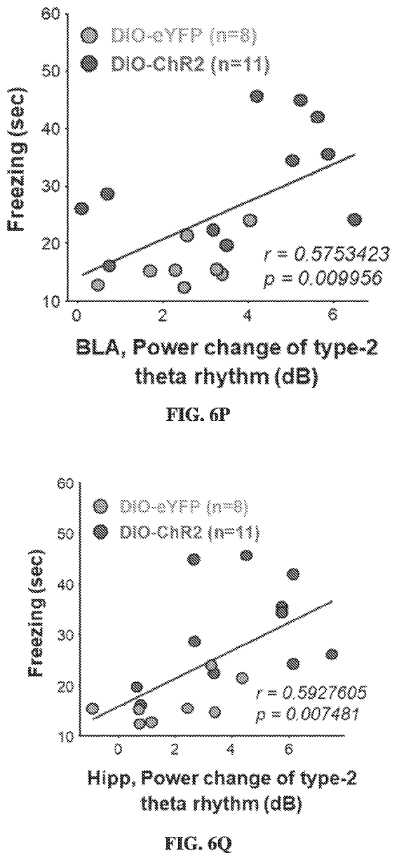

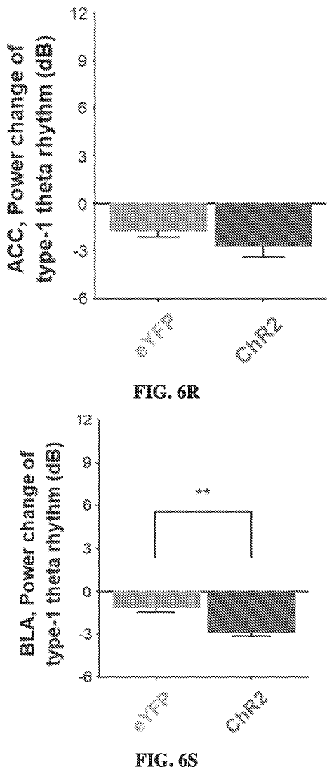

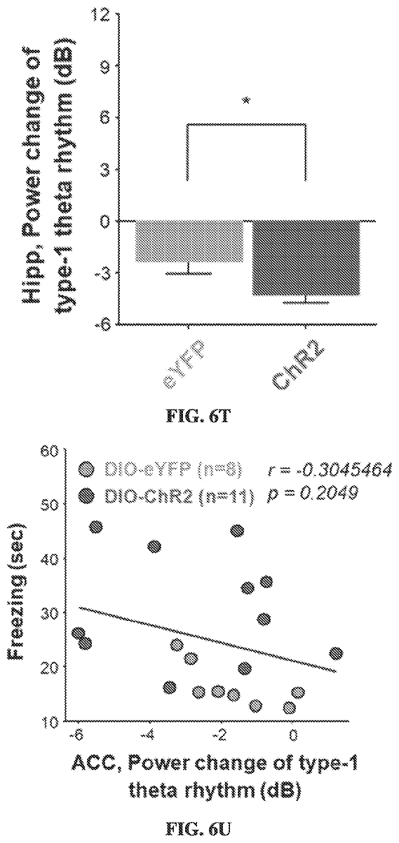

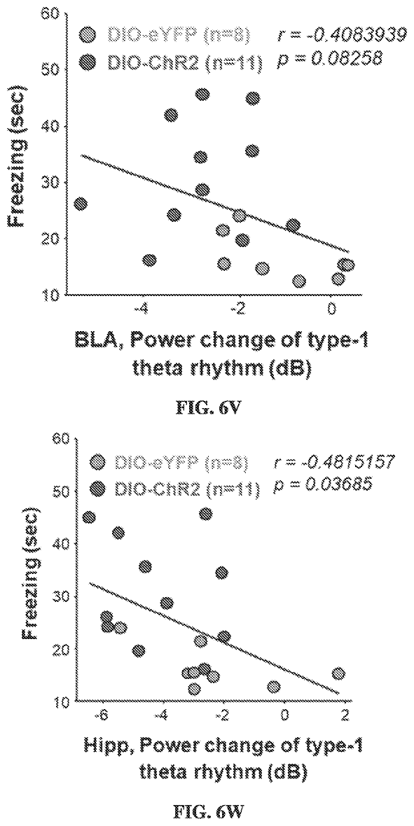

To verify whether experimentally modulating the type-2 hippocampal theta rhythm may enhance observational fear, type-2 hippocampal theta rhythm was modulated by optogenetically activating the MS GABAergic projections to the hippocampus using the procedures described previously by Gangadharan et al. (2016). To this end, AAV5-EF1a-DIO-ChR2-eYFP (DIO-ChR2) was injected into the MS of PV-Cre transgenic mice 4-wk before the observational fear assay (FIG. 6A), and the dorsal fornix was illuminated by a blue laser during observational fear conditioning (FIGS. 6A and 6C). Mice were examined by LFP recording in three brain regions, ACC, BLA and hippocampus, during observational fear (FIG. 6B). Upon optogenetic activation of septo-hippocampal GABAergic fibers during observational fear conditioning (FIG. 6C), DIO-ChR2-injected mice showed significantly enhanced freezing behavior during conditioning compared with AAV5-EF1a-4 DIO-eYFP (DIO-eYFP)-injected control mice (F_{1,17}=9.155, p=0.008, FIG. 6D). The 24-h contextual memory, however, was not significantly enhanced compared with DIO-eYFP-injected mice (P=0.587, t(17)=−0.554, FIG. 6E).

The power change in type-2 hippocampal theta significantly increased by the optogenetic stimulation compared with the control, without a significant change of occurrence and detection of overall 4-12 Hz oscillations. Similar to the previous experiments, power spectrum density revealed enhanced low-frequency theta oscillations in the ACC and BLA both in DIO-eYFP- (FIGS. 6F-H; ACC, F_{8, 112}=5.68, p<12 0.0001; BLA, F_{8, 112}=7.022, P<0.0001; hippocampus, F_{8, 112}=2.391, p<0.05, interaction condition x frequency, two-way repeated measures ANOVA followed by Bonferroni post hoc test), and in DIO-ChR2-injected mice (FIGS. 6I-K, interaction condition x frequency: ACC, F_{8, 160}=14.26, p<0.0001; BLA, F_{8,160}=14.17, P<0.0001; hippocampus, F_{8, 160}=3.521, P<0.0001, two-way repeated measures ANOVA followed by Bonferroni post hoc test). Notably, ChR2-mediated optogenetic stimulation further increased the power change in the 4-8 Hz theta in the hippocampus and ACC during observational fear conditioning (FIGS. 6L-N; ACC, p<0.05, t(17)=2.4211; BLA, p=0.1976, t(17)=1.341; hippocampus, P<0.05, t(17)=2.3425, t-test). The modulation in the power change by optogenetic stimulations was not statistically significant, although showing a tendency, in the BLA. Consistently with the results in FIG. 5 above, the magnitude of the power change in the 4-8 Hz type-2 theta in the ACC, BLA, and hippocampus were positively correlated with freezing behaviors during observational fear (FIGS. 60-Q). On the other hand, power changes in the 8-12 Hz type-1 theta in these brain regions (FIGS. 6R-T) were not correlated with freezing behaviors during observational fear (FIGS. 6U-W). Taken together, these results show that enhanced type-2 hippocampal theta rhythms can enhance observational fear.

### Example 8—Type-2 Theta Rhythms Synchronized in the Network of Hippocampus, ACC, and Amygdala Drive Empathic Fear

This disclosure demonstrates that, in mice, type-2 theta rhythms synchronized in the network consisting of hippocampus, ACC, and amygdala drive empathic fear, without affecting classical fear conditioning. Thus, the rise and fall of the theta rhythm synchrony in the multi-regional circuits precedes the on- and off-set of the freezing behavior respectively. Also, the degree of enhancement of these oscillations predicts the strength of freezing behavior during observational fear, and changing the power of type-2 hippocampal theta oscillations bi-directionally modulate the oscillatory coupling of the three regions and then observational fear.

ACC-BLA projections appear to be functionally heterogeneous, with each involved in distinct fear behaviors. Optogenetic inhibition of ACC-BLA or BLA-ACC excitatory projections impaired observational fear, but did not affect classical contextual fear conditioning (FIG. 2). Therefore, the neural mechanisms underlying observational fear in the reciprocal ACC-BLA connections are different from those involved in classical fear conditioning. Prior experience of a milder foot shock by an observer animal strongly enhances vicarious observational fear learning. In this behavior paradigm, however, optogenetic inhibition of neither the ACC-BLA projections nor the opposite BLA-ACC projections suppressed empathic fear responses during observation of demonstrator's suffering. This contrasts the results that observational fear task carried out with naïve observer mice, in which both ACC-BLA and BLA-ACC circuits were required for observational fear (FIG. 2). This discrepancy suggest that the behaviors revealed by these two different paradigms might be supported by different neural mechanisms. Therefore, observational fear revealed in naïve observer animals should reflect empathic behavior isolated from the complicating issue of socially evoked memory recall by the observer animal of its own prior foot shock experience. Inactivation of ACC-BLA projections enhanced innate fear responses to an olfactory threat stimulus, whereas activation of these projections decreased these responses, supporting the idea that different ACC-BLA projections are involved in diverse cognitive processes leading to fear-related behaviors.

The existence of distinct subtypes of hippocampal theta oscillations and their associations with different brain functions have been shown in humans and rodents. One subset of theta rhythms, called type-1 theta rhythms (8-12 Hz), emerges during voluntary movements. The other subset of theta rhythms, called type-2 theta rhythms (4-8 Hz), are associated with alert immobility, anxiety-related behaviors, innate responses to predator odor, and novelty exploratory behavior. These two forms of theta rhythms are also distinguished from each other by their different pharmacological sensitivities, distinct neural circuits based on optogenetics, and genetic factors involved. The results in this disclosure further strengthen the idea of heterogeneity of theta oscillations in the mammalian brain.

Synchronized theta oscillations between two regions in the brain are believed to be rhythmic neuronal activities for supporting coordinated inter-regional brain communication during cognitive and emotional behaviors in humans and rodents. Nevertheless, the origins and functions of coordinated rhythmic activities at the theta frequency present in the associated structures, such as prefrontal cortex and amygdala, during specific behaviors has yet to be clarified. For example, coordinated 6-12 Hz oscillations in the mPFC and BLA following fear extinction also resemble hippocampal theta rhythm, but its origin remains not determined. Synchronous 4 Hz 7 oscillations in the mPFC and BLA were associated with expression of conditioned fear, which, however, were not dependent on hippocampal theta oscillations. Olfactory inputs can modulate respiration-related rhythmic activity (˜4 Hz) in several brain regions, such as the mPFC, BLA, and hippocampus during conditioned fear-induced freezing behavior. However, these hippocampal respiration-induced oscillations differ from locally generated theta oscillations: hippocampal respiration-coupled rhythm has a unique laminar amplitude profile, and is resistant to atropine. In contrast, this disclosure shows that a synchronized enhancement of the 4-8 Hz rhythm in the ACC-BLA circuit during observational fear is indeed hippocampal type-2 theta, and is strongly associated, temporally as well as quantitatively, with freezing episodes during observational fear. Importantly, this rhythmic synchrony was not required for regular fear conditioning. Thus, type-2 hippocampal theta involved in empathic fear are distinct from respiratory-induced 4 Hz oscillations observed during the classical fear. Also, type-2 theta oscillations in the ACC-BLA circuits represent the cognitive process unique to observational fear, suggesting that the two different fear behaviors, direct versus vicarious, are mediated by distinct neural systems.

Optogenetic inhibition of cell bodies of GABAergic neurons in the medial septum reduced theta activity in all layers of hippocampus, but it had no effect on other hippocampal activities as demonstrated by extensive electrophysiological data. To enhance the selectivity of manipulation further, septo-hippocampal GABAergic fibers in the dorsal fornix was optogenetically targeted, which limited the light stimulation to the septo-hippocampal GABAergic fibers. Anatomically, the septo-hippocampal GABAergic fibers project from the MS to the hippocampus through the dorsal fornix (FIGS. 4A and 6A), and these GABA neurons project throughout the hippocampus. Therefore, optic manipulation via the dorsal fornix is an effective way to modulate the whole septo-hippocampal GABA projections. Because this method manipulates low-frequency theta in both dorsal and ventral hippocampus, it is currently unclear which subregion of hippocampus is important, if any difference exists, for observational fear. Recent studies have suggested a strong involvement of ventral subregion in emotional processing. However, since the theta synchrony with ACC and amygdala during observational fear was found to be fundamentally different from the previously observed oscillatory mechanism during classical fear, it remains to be examined whether observational fear relies on ventral hippocampal functions or depends on other neuronal circuits, such as bidirectional communications between ACC and dorsal hippocampus. It is notable that optogenetic activation of septo-hippocampal GABAergic fibers enhanced observational fear on day-1 conditioning, but did not affect the 24-h contextual memory. This is consistent with the idea that the mechanism underlying the ‘emphatic behavior’ displayed in the day-1 conditioning could be fundamentally different from that underlying the day-2 memory test.

Thus, the disclosure suggests that symptoms of social deficits displayed in diverse neuropsychiatric or neurological conditions could be associated with an impairment in the type-2 theta oscillation in the brain circuits. In other words, an impairment in the type-2 hippocampal theta synchrony could be the universal neural pathology underlying social deficits, which are prevalent in diverse neuropsychiatric or neurological conditions.

PLC-β1 is highly expressed in the cerebral cortex, although it is widely distributed in many brain areas. Furthermore, decreased PLC-β1 expression in several brain regions of patients with schizophrenia, including the dorsolateral prefrontal cortex (DLPFC), which is homologous to the mPFC (including the ACC) in rodents, have suggested a pathogenic involvement of the PLC-β1 downregulation in schizophrenia. Here, using shRNA-mediated silencing of PLC-β1, a mouse model was generated that mimics the decrease of PLC-β1 in the DLPFC of patients with schizophrenia. A behavioral characterization of these model mice revealed that, unlike the phenotypes of the null mutant, PLC-β1^{-/-}, including increased locomotion, decreased anxiety and impaired contextual fear conditioning, ACC-restricted knockdown of PLC-β1 induced impaired observational fear without affecting anxiety or locomotion. In addition, the results disclosed herein distinguish the ACC from the remaining areas of mPFC, i.e., pre-limbic and infra-limbic, for its involvement in affective empathy. Therefore, the neural mechanisms underlying observational fear in the ACC must be different from those underlying the anxiolytic-like behaviors or hyper-locomotion of PLC-β1^{-/-}, the null mutant. Moreover, the strong link between a deficit of theta oscillations and a lack of empathy observed herein could explain social deficits found in human patients with schizophrenia who have reduced PLC-β1. The disclosure confirms that the theta synchronization is the key modulator for the empathic responses.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(1) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

We claim:

1. A method of modulating empathy in a human, comprising: modulating type 2 theta oscillations in a brain region of the human.

2. The method of claim 1, wherein modulating empathy comprises increasing empathy in the human, wherein the human has suboptimal empathy.

3. The method of claim 2, wherein the suboptimal empathy in the human is caused by a psychiatric or neurological condition.

4. The method of any of preceding claims, wherein modulating type 2 theta oscillations comprises optogenetic treatment with or without entrainment.

5. The method of claim 4, wherein the optogenetic treatment comprises stimulating GABAergic neurons.

6. The method of claim 5, wherein the GABAergic neurons are located at the fimbria fornix.

7. The method of claim 5, wherein the GABAergic neurons are located at the septo-hippocampus.

8. The method of any of claims 1 to 3, wherein modulating type 2 theta oscillations comprises genetically modifying neurons in the brain region to directly modulate type-2 theta oscillations.

9. The method claim 8, wherein genetically modifying neurons in the brain region comprises modifying the expression of one or more genes encoding Caᵥ3.2, Caᵥ3.1, CAV3, PLC-β1, and PLC-β4.

10. The method of any of claims 1 to 3, wherein modulating type 2 theta oscillations comprises electrical stimulation of the brain region using one or more electrodes.

11. The method of any of claims 1 to 3, wherein the modulating comprises administering to the human a pharmaceutical drug.

12. The method of claim 11, wherein the pharmaceutical drug is an anticholinesterase agent.

13. The method of claim 12, wherein the anticholinesterase agent is atropine or physostigmine.

14. The method of claim 11, wherein the pharmaceutical drug is an activator of PLC.

15. The method of claim 14, wherein the activator of PLC is an activator of PLC-β1 and/or PLC-β4.

16. The method of claim 14 or 15, wherein the activator of PLC is m3M3FBS (2,4,6-trimethyl-N-(meta-3-trifluoromethyl-phenyl)-benzenesulfonamide) or thapsigargin.

17. The method of any of claims 1 to 3, wherein the brain region is: hippocampus, septo-hippocampus, anterior cingulate cortex (ACC), basolateral amygdala (BLA), midline thalamus, insulate regions, medial septum (MS), or fimbria fornix.

18. The method of any of claims 1 to 3, wherein the type 2 theta oscillations are synchronized in the hippocampus, ACC, or BLA.

19. The method of claim 3, wherein the psychiatric or neurological condition is: Alzheimer's disease, autism spectrum disorder, dementia, addiction, depression, anxiety, bipolar disorder, schizophrenia, attention-deficit hyperactivity disorder (ADHD), alexithymia, obsessive-compulsive disorder (OCD), post-traumatic stress disorder (PTSD), psychopathy, Parkinson's disease, or epilepsy.

20. The method of claim 19, wherein the psychiatric or neurological condition is selected from the group consisting of: autism spectrum disorder, dementia, and addiction.

21. The method of any of claims 1 to 3, wherein the modulating is increasing type 2 theta oscillations.

22. The method of claim 1, wherein the modulating is decreasing type 2 theta oscillations.

23. The method of any of claims 1 to 3, wherein the modulating of type 2 theta oscillations does not induce modulation of type 1 theta oscillations.

24. A system for modulating empathy in a human, comprising: a device configured to modulate type 2 theta oscillations in a brain region of the human.

25. The system of claim 24, wherein the device is configured to increase empathy in a human having suboptimal empathy.

26. The system of claim 24 or 25, wherein the device is an optogenetic device comprising a light source operationally coupled to a light transmitter configured and positioned to deliver light to the brain region.

27. The system of claim 24 or 25, wherein the device is an electrical stimulation device comprising an electrical current generator operationally coupled to at least one electrode configured and positioned to deliver electrical stimulation to the brain region.

28. The system of claim 27, wherein the system comprises two electrodes.

29. The system of claim 24 or 25, wherein the brain region is: hippocampus, septo-hippocampus, ACC, BLA, midline thalamus, insulate regions, medial septum, fimbria fornix, amygdala, or a combination thereof.
